SEQUENCE LISTING (9 sequences)

SEQ ID NO: 1 - DNA, 19 bp, synthetic construct
  misc_feature 1..19: note = Synthetic: Forward
  source 1..19: mol_type = other DNA
ctcgtggtggacttctctc

SEQ ID NO: 2 - DNA, 18 bp, synthetic construct
  misc_feature 1..18: note = Synthetic: Reverse
  source 1..18: mol_type = other DNA
cagcaggatgaagaggaa

SEQ ID NO: 3 - DNA, 25 bp, synthetic construct
  misc_feature 1..25: note = Synthetic: Probe
  source 1..25: mol_type = other DNA
ccaacctccaatcactcaccaacct

SEQ ID NO: 4 - DNA, 18 bp, synthetic construct
  misc_feature 1..18: note = Synthetic: Forward
  source 1..18: mol_type = other DNA
cacctctgcctaatcatc

SEQ ID NO: 5 - DNA, 20 bp, synthetic construct
  misc_feature 1..20: note = Synthetic: Reverse
  source 1..20: mol_type = other DNA
ggaaagaagtcagaaggcaa

SEQ ID NO: 6 - DNA, 25 bp, synthetic construct
  misc_feature 1..25: note = Synthetic: Probe
  source 1..25: mol_type = other DNA
tgtccyactgttcaagcctccaagc

SEQ ID NO: 7 - DNA, 19 bp, synthetic construct
  misc_feature 1..19: note = Synthetic: Forward
  source 1..19: mol_type = other DNA
taggaggctgtaggcataa

SEQ ID NO: 8 - DNA, 19 bp, synthetic construct
  misc_feature 1..19: note = Synthetic: Reverse
  source 1..19: mol_type = other DNA
cacagcttggaggcttgaa

SEQ ID NO: 9 - DNA, 30 bp, synthetic construct
  misc_feature 1..30: note = Synthetic: Probe
  source 1..30: mol_type = other DNA
tgcaactttttcacctctgcctaatcatct

(19) United States
(12) Patent Application Publication
(10) Pub. No.: US 2025/0137077 A1
(43) Pub. Date: May 1, 2025

(54) PAN-GENOTYPIC ASSAY AND RELATED ASPECTS FOR THE QUANTITATIVE IDENTIFICATION OF cccDNA-DERIVED OR INTEGRATED DNA-DERIVED HEPATITIS B SURFACE ANTIGEN

(71) Applicant: THE JOHNS HOPKINS UNIVERSITY, Baltimore, MD (US)

(72) Inventors: Ashwin BALAGOPAL, Columbia, MD (US); Chloe L. THIO, Baltimore, MD (US); Tanner GRUDDA, Baltimore, MD (US); Hyon HWANG, Lutherville, MD (US)

(21) Appl. No.: 18/835,397

(22) PCT Filed: Feb. 8, 2023
(86) PCT No.: PCT/US2023/062205

§ 371 Date: Aug. 2, 2024

Related U.S. Application Data
(60) Provisional application No. 63/309,216, filed on Feb. 11, 2022.

(51) Int. Cl. C12Q 1/70; C12Q 1/6851

(52) U.S. Cl. CPC C12Q 1/706; C12Q 1/6851; C12Q 2600/158

(57) ABSTRACT

Provided herein are methods of detecting sources of hepatitis B virus (HBV) surface antigens (HbsAgs) in samples. Some exemplary embodiments include amplifying HBV transcript nucleic acids in or from a sample and quantifying an amount of HBV transcript nucleic acids transcribed from HBV cccDNA and an amount of HBV transcript nucleic acids transcribed from the HBV iDNA. Related systems and other aspects are also provided.

### CROSS-REFERENCE TO RELATED APPLICATIONS

This application is the national stage entry of International Patent Application No. PCT/US2023/062205, filed on Feb. 8, 2023, and published as WO 2023/154742 A1 on Aug. 17, 2023, which claims the benefit of U.S. Provisional Patent Application Ser. No. 63/309,216, filed on Feb. 11, 2022, which are hereby incorporated by reference in their entireties.

### STATEMENT REGARDING FEDERALLY SPONSORED RESEARCH OR DEVELOPMENT

This invention was made with government support under grants AI138810, AI157760, and AI165166 awarded by the National Institutes of Health. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Feb. 3, 2023, is named “0184.0193-PCT.xml” and is 12,369 bytes in size. The sequence listing contained in this .XML file is part of the specification and is hereby incorporated by reference herein in its entirety.

### BACKGROUND

Over 250 million people are chronically infected with hepatitis B virus (HBV), the leading cause of hepatocellular carcinoma and cirrhosis worldwide. Up to 28% of people living with HIV-1 (PLWH) have chronic HBV (CHB) and are at risk for worse outcomes; thus, discovering an HBV cure is imperative for persons with and without HIV. The NIH defines HBV cure as hepatitis B surface antigen (HBsAg) loss and also states that “silencing cccDNA (covalently closed circular DNA) is considered essential for cure”. However, HBsAg loss may be difficult to achieve since HBsAg derives from two sources: 1) covalently closed circular DNA (cccDNA), the stable genomic template for all viral mRNAs (FIG. 1A) and 2) integrated HBV DNA (iDNA), a portion of the HBV genome that is integrated into the human genome. Determining which of these sources is contributing to the circulating HBsAg is essential since cure strategies would need to be targeted to eliminating the source(s) of HBsAg in individuals with chronic hepatitis B. Currently, it is not possible to distinguish which of these sources HBsAg derives from in an individual. As potential emerging therapies for HBV are being studied, it is of importance to distinguish whether and to what degree HBsAg derives from cccDNA or iDNA. This is because some newer therapies may target HBsAg derived from cccDNA exclusively, others may target HBsAg derived from iDNA exclusively, and others may target both.

Accordingly, there is a need for methods and related aspects to determine how much HBsAg derives from cccDNA versus iDNA.

### SUMMARY

The present disclosure relates, in certain aspects, to methods and systems of use in detecting and quantifying sources of hepatitis B virus (HBV) surface antigen (HBsAg) in samples. In some of these applications, the methods and related aspects disclosed herein are used to monitor a course of therapy in subjects infected with HBV and to stratify subjects into different patient populations to help refine therapeutic decision-making for those subjects. These and other aspects will be apparent upon a complete review of the present disclosure, including the accompanying figures.

In some aspects, the present disclosure provides a method of detecting sources of hepatitis B virus (HBV) surface antigen (HBsAg) in a sample by focusing on S transcripts (mRNAs) that are then translated into the HBsAg protein. The method includes amplifying HBV transcript nucleic acids in or from the sample using at least two sets of nucleic acid primers and nucleic acid probes (e.g., in a multiplexed reaction mixture; Table 1; FIG. 1C). A first set of nucleic acid primers and nucleic acid probes targets the middle of the HBV genomic region that is also common to both HBV nucleic acids transcribed from HBV covalently closed circular DNA (cccDNA) and from HBV integrated DNA (iDNA) that is termed herein as the mid-HBV amplicon (FIG. 1C). A second set of nucleic acid primers and nucleic acid probes targets a second portion of the HBV genomic region, the 3′ end of HBV genome, that is common to HBV transcript nucleic acids transcribed from HBV cccDNA and not from HBV iDNA, called the 3′-HBV amplicon, and/or a third set of nucleic acid primers and nucleic acid probes targets a third HBV genomic region, the 5′ end of the HBV genome, that is used to distinguish full-genome transcripts from those that only target the S transcript called the 5′-HBV amplicon to produce amplification data that comprises one or more detectable signal levels detected as the HBV transcript nucleic acids are amplified. The method also includes quantifying a first amount of the HBV transcript nucleic acids transcribed from the HBV cccDNA and a second amount of the HBV transcript nucleic acids transcribed from the HBV iDNA from the amplification data, thereby detecting and quantifying the contribution of each source of the HBsAg in a sample and distinguishing them from other HBV transcripts (FIG. 1B).

In some embodiments, the method further includes obtaining the sample from a subject. In some embodiments, the sample comprises hepatocellular tissue, serum, and/or plasma. In some embodiments, the methods include amplifying the HBV transcript nucleic acids in or from the sample using a droplet digital polymerase chain reaction (ddPCR) technique or a qPCR technique (e.g., qRT-PCR or the like). In some embodiments, the method further comprises using the method to stratify subjects into different patient populations based upon the quantified amounts of the mid-HBV amplicons and the 3′-HBV amplicons and/or the 5′-HBV amplicons for a given subject. In some embodiments, the method comprises administering one or more therapies to the subject based at least in part on a ratio of the quantified amounts of the mid-HBV amplicons and the 3′-HBV amplicons and/or the 5′-HBV amplicons. In some embodiments, the method comprises discontinuing administering one or more therapies to the subject based at least in part on a ratio of the quantified amounts of the mid-HBV amplicons and the 3′-HBV amplicons and/or the 5′-HBV amplicons. In some embodiments, the method further comprises correlating the quantified amounts of the mid-HBV amplicons and the 3′-HBV amplicons and/or the 5′-HBV amplicons with plasma levels of one or more of the Large, Middle, Small HBsAg in the subject. In some embodiments, the method further comprises quantifying the amounts of the mid-HBV amplicons and the 3′-HBV amplicons and/or the 5′-HBV amplicons from multiple samples obtained from the subject at different time points.

In some embodiments, the sets of nucleic acid primers and nucleic acid probes are selected from the group consisting of: a set of nucleic acids that targets a 5′ end of pre-genomic HBV RNA (5′-HBV amplicon); a set of nucleic acids that targets all HBV transcripts corresponding to the middle of the HBV genome (mid-HBV amplicon); and a set of nucleic acids that targets a 3′ end of HBV transcript nucleic acids that are common to all HBV transcript nucleic acids that are transcribed from cccDNA (3′-HBV amplicon). In some embodiments, the sets of nucleic acid primers and nucleic acid probes are selected from the group consisting of: a set of nucleic acids having nucleotide sequences of SEQ ID NOs: 1, 2, and 3; a set of nucleic acids having nucleotide sequences of SEQ ID NOs: 4, 5, and 6; and a set of nucleic acids having nucleotide sequences of SEQ ID NOs: 7, 8, and 9 (Table 1). In some embodiments, the subject has chronic HBV infection and wherein a detected decline in total quantitative HBsAg (qHBsAg) of >0.5 log₁₀ international units/mL in the subject indicates that the HBV transcript nucleic acids are transcribed primarily from the HBV cccDNA. In some embodiments, the subject has chronic HBV infection and wherein an absence of a detected decline in total quantitative HBsAg (qHBsAg) in the subject indicates that the HBV transcript nucleic acids are transcribed primarily from the HBV iDNA.

In some aspects, the present disclosure provides a system that includes a nucleic acid amplification component configured to amplify HBV transcript nucleic acids in or from a sample using at least two sets of nucleic acid primers and nucleic acid probes in which a first set of nucleic acid primers and nucleic acid probes targets the middle of the HBV genomic region that is also common to both HBV nucleic acids transcribed from HBV covalently closed circular DNA (cccDNA) and from HBV integrated DNA (iDNA), and in which a second set of nucleic acid primers and nucleic acid probes a second portion of the HBV genomic region, the 3′ end of HBV genome, that is common to HBV transcript nucleic acids transcribed from HBV cccDNA and not from HBV iDNA, called the 3′-HBV amplicon, and/or in which a third set of nucleic acid primers and nucleic acid probes targets a third HBV genomic region, the 5′ end of the HBV genome, that is used to distinguish full-genome transcripts from those that only target the S transcript called the 5′-HBV amplicon to produce amplification data that comprises one or more detectable signal levels detected as the HBV transcript nucleic acids are amplified. The system also includes a controller operably connected to the nucleic acid amplification component, which controller comprises, or is capable of accessing, computer readable media comprising non-transitory computer-executable instructions which, when executed by at least one electronic processor, perform at least: quantifying amounts of the mid-HBV amplicons and the 3′-HBV amplicons and/or the 5′-HBV amplicons from the amplification data to thereby detect sources of hepatitis B virus (HBV) surface antigen (HBsAg) in the sample.

In some embodiments, wherein the sample comprises hepatocellular tissue, serum, and/or plasma. In some embodiments, the nucleic acid amplification component is configured to perform a droplet digital polymerase chain reaction (ddPCR) technique or a qPCR technique (e.g., qRT-PCR or the like). In some embodiments, the computer-executable instructions which, when executed by the at least one electronic processor, further perform at least: stratifying subjects into different patient populations based upon the quantified amounts of the mid-HBV amplicons and the 3′-HBV amplicons and/or the 5′-HBV amplicons for a given subject. In some embodiments, the computer-executable instructions which, when executed by at least one electronic processor, further perform at least: generating a report recommending administering one or more therapies to the subject based at least in part on a ratio of the quantified amounts of the mid-HBV amplicons and the 3′-HBV amplicons and/or the 5′-HBV amplicons. In some embodiments, the computer-executable instructions which, when executed by the at least one electronic processor, further perform at least: generating a report recommending discontinuing administering one or more therapies to the subject based at least in part on a ratio of the quantified amounts of the mid-HBV amplicons and the 3′-HBV amplicons and/or the 5′-HBV amplicons. In some embodiments, the computer-executable instructions which, when executed by the at least one electronic processor, further perform at least: correlating the quantified amounts of the mid-HBV amplicons and the 3′-HBV amplicons and/or the 5′-HBV amplicons with serum or plasma levels of one or more of the HBsAgs in the subject.

In some embodiments, the sets of nucleic acid primers and nucleic acid probes are selected from the group consisting of: a set of nucleic acids that targets a 5′ end of pre-genomic HBV RNA (5′-HBV amplicon); a set of nucleic acids that targets all types of HBV transcript nucleic acids (mid-HBV amplicon); and a set of nucleic acids that targets a 3′ end of HBV transcript nucleic acids that are common to all HBV transcript nucleic acids that are transcribed from cccDNA but not iDNA (3′-HBV amplicon). In some embodiments, the sets of nucleic acid primers and nucleic acid probes are selected from the group consisting of: a set of nucleic acids having nucleotide sequences of SEQ ID NOs: 1, 2, and 3; a set of nucleic acids having nucleotide sequences of SEQ ID NOs: 4, 5, and 6; and a set of nucleic acids having nucleotide sequences of SEQ ID NOs: 7, 8, and 9 (Table 1).

### BRIEF DESCRIPTION OF THE DRAWINGS

The accompanying drawings, which are incorporated in and constitute a part of this specification, illustrate certain embodiments, and together with the written description, serve to explain certain principles of the methods, devices, kits, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1A-1C schematically show that S transcripts (mRNAs) from cccDNA and iDNA differ in 3′ viral termination. Shown are the canonical HBV transcripts terminating at a common region when originating from cccDNA (A) and fusing to the human genome at varying points depending on the integration process as represented by the bracket above the right portion of (C). Shaded portions indicate the locations of ddPCR amplicons to quantify mid-HBV S (middle box with dots) and 3′-HBV S (box on right side with vertically dashed lines) (designed to be after the majority of 3′-S transition points of iDNA. Shown is a simulated ddPCR flow plot (B) from liver tissue that will be used to discriminate S mRNAs that derive from cccDNA and S mRNAs that derive from iDNA, using multiplexed primers/probes to simultaneously detect the mid- and 3′ ends of S mRNAs. cccDNA-derived S mRNAs will be detected by both mid-HBV and 3′-HBV amplicons, while iDNA-derived S mRNAs will only be detected by the mid-HBV amplicon.

FIG. 2 shows the efficiency of each assay compared to one another. A purified DNA product was weighed and the copy/uL was calculated based on sequence before being run with the 5′-HBV, mid-HBV, and 3′-HBV assay on ddPCR. The assays are equally efficient across a range of dilutions ideal for multiplexing samples.

FIG. 3 demonstrates the specificity of the assay as the mid-HBV amplicon is detectable across a range of dilutions transcripts deriving from iDNA exclusively while the 3′-HBV amplicon is undetectable.

FIG. 4 demonstrates the detectability of the mid-HBV and 3′-HBV amplicons in a cccDNA transcribing sample. This in vivo production model cannot produce iDNA and its subsequent transcripts, but can produce X transcripts in abundance, something not thought to occur in natural infection.

FIG. 5 shows the number of amplicons detectable for the mid-HBV and 3′-HBV assays in single hepatocytes measured by the assay. Calculations were applied to these values to bin cells as transcribing from cccDNA, iDNA, or a mixture of cccDNA and iDNA. We were able to detect 5/40 cells transcribing in abundance from iDNA and 2/40 from cccDNA exclusively demonstrating the assay's ability to classify a cell's transcriptional regulation during chronic HBV.

FIG. 6 is a flow chart that schematically shows exemplary method steps of detecting sources of hepatitis B virus (HBV) surface antigen (HBsAg) in a sample according to some aspects disclosed herein.

FIG. 7 is a schematic diagram of an exemplary system suitable for use with certain embodiments.

FIG. 8 is a plot showing quantified serum HBsAg levels and iDNA- and cccDNA-derived S in bulk liver in 16 people at two time points separated by 3-4 years on HBV-active ART. Persons with <0.5 log₁₀ IU/mL declines in serum HBsAg had S transcription predominantly from iDNA (left), whereas persons with ≥0.5 log₁₀ IU/mL decline had S transcription chiefly from cccDNA (right). Symbols are the iDNA transcriptional index (iDNA transcript #/cccDNA transcript #) at each liver biopsy for each person.

FIG. 9 is a plot demonstrating the ability of this assay to relate viral transcription in the liver to HBsAg detection in the periphery. This proof of concept may become relevant for researchers, practitioners, and clinicians when deciding treatments and responses for patients.

FIG. 10 schematically shows an HBV transcriptional map and multiplex ddPCR assay targets. Horizontal lines depict HBV transcripts derived from cccDNA or iDNA (labeled). The variable chimeric viral/host region is shown as hashed lines at the 3′ end of iDNA-derived transcripts. The dashed vertical line is the EcoRI cut site for orientation. Solid vertical lines show the locations of the DR1, DR2, and poly A signal (PAS) regions for reference. Individual targeted assays for each component of the multiplex ddPCR are shown as vertical boxes spanning the shaded regions at the 5′, middle, and 3′ ends of HBV transcripts.

FIG. 11 is a plot showing serum HBsAg over time. Serum HBsAg declines are shown for each person between the time of their liver biopsies. Each person (n=16) is represented by a unique line. The dotted lines indicate people who achieved a >log₁₀ 0.5 IU/mL decline between biopsies, while unbroken lines indicate the people who had smaller HBsAg declines.

FIG. 12 is a plot showing HBV transcriptional activity and serum HBsAg levels. Total HBV transcriptional activity in liver was measured by the mid-HBV assay that targets all HBV transcripts (except low abundance 0.7 kb transcripts) and is located in the middle of the S ORF. Quantities were adjusted for the number of cells in each sample using ERV-3 quantities. HBV transcriptional activity in liver was correlated with contemporaneous serum HBsAg levels. Each point represents one person at each biopsy (n=32) and the black trendline was calculated using simple linear regression while the shaded region depicts the standard error along the trendline. Black points depict the four individuals with >log₁₀ 0.5 IU/mL declines in serum HBsAg at biopsy 1 with lines drawn to biopsy 2 for each person. Spearman's correlation coefficient is shown with its p-value and the shape of points (circles or triangles) corresponds to HBeAg status at the time of biopsy, as indicated in the legend.

FIG. 13 is a plot showing HBsAg decline during NUC treatment and iDNA-derived transcription. Boxplots represent the summarized serum HBsAg levels at the time of each biopsy for people who achieve a >0.5 log₁₀ IU/mL decline (left panel, n=4) and those who had 50.5 log₁₀ IU/mL (right panel, n=12). The absolute HBsAg levels are shown on the leftward y-axis. Points corresponding to the contemporaneous liver measurements of the iDNA transcriptional index (iDNA TI) from the same people are overlaid and quantified by the rightward y-axis (n=32). A horizontal cutoff line indicates a sample for which the majority of HBV transcripts derive from iDNA since an iDNA TI>2 indicates that more transcripts derive from iDNA than from cccDNA. Bx1-Biopsy 1; Bx2-Biopsy 2.

FIGS. 14A and 14B are plots showing iDNA transcription and serum HBsAg in relation to pgRNA TI (surrogate for cccDNA-derived transcription). Each point represents each person at each biopsy with the shape of point indicating HBeAg status at the time of biopsy (n=32). Trendlines were calculated using a simple linear regression, with shading indicating the standard error around the lines. (A) Spearman's correlation coefficient shows the relationship between iDNA TI and pgRNA TI, a measure of cccDNA transcription. (B) Serum HBsAg levels are shown correlated with the pgRNA TI in two components. To the right are shown points and a simple linear regression from people with ≥100 pgRNA transcripts/cccDNA molecule wherein all people had been NUC-suppressed for <5 years. To the left are shown people<100 pgRNA transcripts/cccDNA molecule and the majority of these people have been NUC-suppressed for ≥5 years. Spearman's correlation coefficients and p-values corresponding to each group are shown under each component.

FIGS. 15A and 14B are plots showing HBV serology and single-cell viral transcriptional landscapes from three representative people over time. (A) Serum HBV DNA (darker trace) and serum HBsAg (lighter trace) levels between biopsies are represented for each person who underwent single cell examination (n=3). All available time points are shown and open circles are those that were below the limit of detection for each assay. Vertical dashed lines indicate when biopsies were taken from each person. (B) Each point represents a single hepatocyte after QC exclusion. Grids are arrayed in the xy plane to reflect their position in a contiguous section of tissue. Three representative people are shown at each biopsy. The size of each point corresponds with the quantity of HBV transcripts/cell, as measured by the mid-HBV assay, and open points indicate that no HBV transcription was detected. Different shades indicate the source of viral transcripts in cells, as specified in the legend and determined by the iDNA TI. Some cells were found to have cccDNA-derived transcription but had undetectable S transcripts: these were shaded lighter and shown as the smallest points. All cells that were deemed analyzable (because they passed QC by having sufficient total RNA) are depicted, irrespective of their infection or viral transcriptional status.

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, systems, computer readable media, and component parts, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Administering: As used herein, the terms “administering” and “administration” refer to any method of providing a pharmaceutical preparation or other treatment to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

Amplifying: As used herein, “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using RT-PCR is a form of amplification. Furthermore, the generation of multiple RNA molecules from a single DNA molecule during the process of transcription is also a form of amplification.

Detect: As used herein, “detect,” “detecting,” or “detection” refers to an act of determining the existence or presence of one or more target nucleic acids (e.g., nucleic acids having targeted mutations or other markers) in a sample.

Detectable Signal: As used herein, “detectable signal” refers to signal output at an intensity or power sufficient to be detected in a given detection system. In certain embodiments, a detectable signal is emitted from a label (e.g., a fluorescent label or the like) associated with a given primer nucleic acid and/or probe nucleic acid.

Exonuclease Probe: As used herein, “exonuclease probe” refers to a labeled oligonucleotide that is capable of producing a detectable signal change upon being cleaved. To illustrate, in certain embodiments an exonuclease probe is a 5′-nuclease probe comprising two labeling moieties and emits radiation of increased intensity after one of the labels is cleaved or otherwise separated from the oligonucleotide. In some of these embodiments, for example, the 5-nuclease probe is labeled with a 5′ terminus quencher moiety and a reporter moiety at the 3′ terminus of the probe. In certain embodiments, 5-nuclease probes are labeled at one or more positions other than, or in addition to, these terminal positions. When the probe is intact, energy transfer typically occurs between the labeling moieties such that the quencher moiety at least in part quenches the fluorescent emission from the acceptor moiety. During an extension step of a polymerase chain reaction, for example, a 5′-nuclease probe bound to a template nucleic acid is cleaved by the 5′ to 3′ nuclease activity of, e.g., a Taq polymerase or another polymerase having this activity such that the fluorescent emission from the acceptor moiety is no longer quenched. To further illustrate, in certain embodiments 5′-nuclease probes include regions of self-complementarity such that the probes are capable of forming hairpin structures under selected conditions. In these embodiments, 5′-nuclease probes are also referred to herein as “hairpin probes.”

Hairpin Probe: As used herein, “hairpin probe” refers to an oligonucleotide that can be used to effect target nucleic acid detection and that includes at least one region of self-complementarity such that the probe is capable of forming a hairpin or loop structure under selected conditions. Typically, hairpin probes include one or more labeling moieties. In one exemplary embodiment, quencher moieties and reporter moieties are positioned relative to one another in the hairpin probes such that the quencher moieties at least partially quench light emissions from the reporter moieties when the probes are in hairpin confirmations. In contrast, when the probes in these embodiments are not in hairpin confirmations (e.g., when the probes are hybridized with target nucleic acids), light emissions the acceptor reporter moieties are generally detectable. Hairpin probes are also known as molecular beacons in some of these embodiments. Hairpin probes can also function as 5′-nuclease probes or hybridization probes in certain embodiments.

Hybridization Probe: As used herein, “hybridization probe” refers an oligonucleotide that includes at least one labeling moiety that can be used to effect target nucleic acid detection. In some embodiments, hybridization probes function in pairs. In some of these embodiments, for example, a first hybridization probe of a pair includes at least one donor moiety at or proximal to its 3′-end, while the second hybridization probe of the pair includes at least one acceptor moiety (e.g., LC-Red 610, LC-Red 640, LC-Red 670, LC-Red 705, JA-270, CY5, or CY5.5) at or proximal to its 5′-end. The probes are typically designed such that when both probes hybridize with a target or template nucleic acid (e.g., during a PCR), the first hybridization probe binds to the 5′-end side or upstream from the second hybridization probe and within sufficient proximity for energy transfer to occur between the donor and acceptor moieties to thereby produce a detectable signal. Typically, the second hybridization probe also includes a phosphate or other group on its 3′-end to prevent extension of the probe during a PCR.

Label: As used herein, “label” refers to a moiety attached (covalently or non-covalently), or capable of being attached, to a molecule, which moiety provides or is capable of providing information about the molecule (e.g., descriptive, identifying, etc. information about the molecule). Exemplary labels include donor moieties, acceptor moieties, fluorescent labels, non-fluorescent labels, calorimetric labels, chemiluminescent labels, bioluminescent labels, radioactive labels, mass-modifying groups, antibodies, antigens, biotin, haptens, and enzymes (including, e.g., peroxidase, phosphatase, etc.).

Mixture: As used herein, “mixture” refers to a combination of two or more different components.

Nucleic Acid: As used herein, “nucleic acid” refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., bromodeoxyuridine (BrdU)), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA, cfDNA, ctDNA, dsRNA, ssRNA, mRNA, lncRNA, spRNA, or any combination thereof.

Primer Nucleic Acid: As used herein, “primer nucleic acid” or “primer” refers to a nucleic acid that can hybridize to a target or template nucleic acid and permit chain extension or elongation using, e.g., a nucleotide incorporating biocatalyst, such as a polymerase under appropriate reaction conditions. A primer nucleic acid is typically a natural or synthetic oligonucleotide (e.g., a single-stranded oligodeoxyribonucleotide). Although other primer nucleic acid lengths are optionally utilized, they typically comprise hybridizing regions that range from about 8 to about 100 nucleotides in length. Short primer nucleic acids generally require cooler temperatures to form sufficiently stable hybrid complexes with template nucleic acids. A primer nucleic acid that is at least partially complementary to a subsequence of a template nucleic acid is typically sufficient to hybridize with the template for extension to occur. A primer nucleic acid can be labeled, if desired, by incorporating a label detectable by, e.g., spectroscopic, photochemical, biochemical, immunochemical, chemical, or other techniques. To illustrate, useful labels include donor moieties, acceptor moieties, quencher moieties, radioisotopes, electron-dense reagents, enzymes (as commonly used in performing ELISAs), biotin, or haptens and proteins for which antisera or monoclonal antibodies are available. Many of these and other labels are described further herein and/or are otherwise known in the art. One of skill in the art will recognize that, in certain embodiments, primer nucleic acids can also be used as probe nucleic acids.

Probe Nucleic Acid: As used herein, “probe nucleic acid” or “probe” refers to a labeled or unlabeled oligonucleotide capable of selectively hybridizing to a target or template nucleic acid under suitable conditions. Typically, a probe is sufficiently complementary to a specific target sequence contained in a nucleic acid sample to form a stable hybridization duplex with the target sequence under a selected hybridization condition, such as, but not limited to, a stringent hybridization condition. A hybridization assay carried out using a probe under sufficiently stringent hybridization conditions permits the selective detection of a specific target sequence. The term “hybridizing region” refers to that region of a nucleic acid that is exactly or substantially complementary to, and therefore capable of hybridizing to, the target sequence. For use in a hybridization assay for the discrimination of single nucleotide differences in sequence, the hybridizing region is typically from about 8 to about 100 nucleotides in length. Although the hybridizing region generally refers to the entire oligonucleotide, the probe may include additional nucleotide sequences that function, for example, as linker binding sites to provide a site for attaching the probe sequence to a solid support. A probe of the invention is generally included in a nucleic acid that comprises one or more labels (e.g., donor moieties, acceptor moieties, and/or quencher moieties), such as exonuclease probe (e.g., a 5′-nuclease probe), a hybridization probe, a fluorescent resonance energy transfer (FRET) probe, a hairpin probe, or a molecular beacon, which can also be utilized to detect hybridization between the probe and target nucleic acids in a sample. In some embodiments, the hybridizing region of the probe is completely complementary to the target sequence. However, in general, complete complementarity is not necessary (i.e., nucleic acids can be partially complementary to one another); stable hybridization complexes may contain mismatched bases or unmatched bases. Modification of the stringent conditions may be necessary to permit a stable hybridization complex with one or more base pair mismatches or unmatched bases. Stability of the target/probe hybridization complex depends on a number of variables including length of the oligonucleotide, base composition and sequence of the oligonucleotide, temperature, and ionic conditions. One of skill in the art will recognize that, in general, the exact complement of a given probe is similarly useful as a probe. One of skill in the art will also recognize that, in certain embodiments, probe nucleic acids can also be used as primer nucleic acids.

Quantitation Cycle: As used herein, “quantitation cycle,” “Cq,” “cycle threshold,” “Cp,” or “Ct” refers to the cycle or point in a given amplification reaction at which the detectable signal intensity or power is above background noise levels.

Reaction Mixture: As used herein, “reaction mixture” refers a mixture that comprises molecules that can participate in and/or facilitate a given reaction or assay. To illustrate, an amplification reaction mixture generally includes a solution containing reagents necessary to carry out an amplification reaction, and typically contains primers, a biocatalyst (e.g., a nucleic acid polymerase, a ligase, etc.), dNTPs, and a divalent metal cation in a suitable buffer. A reaction mixture is referred to as complete if it contains all reagents necessary to carry out the reaction, and incomplete if it contains only a subset of the necessary reagents. It will be understood by one of skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of skill in the art that reaction components are packaged separately for commercialization and that useful commercial kits may contain any subset of the reaction or assay components.

Sample: As used herein, “sample” refers to a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a nucleic acid), which is assayed as described herein. A sample may also be any body fluid or excretion (for example, but not limited to, blood, urine, stool, saliva, tears, bile) that contains cells, cell components, or non-cellular fractions.

Subject: As used herein, “subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.” For example, a subject can be an individual who has been diagnosed with having a respiratory disease, disorder, or condition, is going to receive a therapy for a respiratory disease, disorder, or condition, and/or has received at least one therapy for a respiratory disease, disorder, or condition.

System: As used herein, “system” in the context of analytical instrumentation refers a group of objects and/or devices that form a network for performing a desired objective.

Target: As used herein, “target” refers to a biomolecule (e.g., a nucleic acid, etc.), or portion thereof, that is to be amplified, detected, and/or otherwise analyzed.

Treatment: As used herein, “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human.

Value: As used herein, “value” generally refers to an entry in a dataset that can be anything that characterizes the feature to which the value refers. This includes, without limitation, numbers, words or phrases, symbols (e.g., + or −) or degrees.

### DETAILED DESCRIPTION

An objective present disclosure is to develop a tool identify the different origins of total hepatitis B surface antigen (HBsAg) production in people with chronic hepatitis B. Over 290 million people are chronically infected with HBV, the leading cause of hepatocellular carcinoma and cirrhosis worldwide. Up to 28% of people living with HIV-1 (PLWH) have chronic HBV (CHB) and are at risk for worse outcomes; thus, discovering an HBV cure is imperative for people with and without HIV. The NIH defines HBV cure as HBsAg loss after completion of therapy and states that “silencing cccDNA (covalently closed circular DNA) is considered essential for cure”. However, HBsAg loss may be difficult to achieve since HBsAg derives from two sources: 1) cccDNA, the stable genomic template for all viral mRNAs and 2) integrated HBV DNA (iDNA), a portion of the HBV genome that is integrated into the human genome. To achieve HBsAg loss, it is important to understand the contributions from these two sources to HBsAg, which is contained in the common C-terminal domain of the Large (L-HBs), Middle (M-HBs), and Small (S-HBs) envelope proteins. The S-HBs is the most abundant subset.

cccDNA is transcribed into five viral mRNAs that share a common 3′ sequence and poly-A tail, including a 2.4 kb mRNA that is translated into L-HBs and a 2.1 kb mRNA that is translated into M- and S-HBs through different open reading frames. In contrast, iDNA produces multiple mRNAs that differ from cccDNA-derived mRNAs by transitioning to human sequences upstream of the common viral 3′ poly-A tail, and these iDNA-derived S transcripts (mRNAs) are translated into S-HBs.

We used droplet digital PCR (ddPCR) to reveal that cccDNA transcription is diminished nearly 100-fold during nucelos(t)ide analogue (NUC) treatment. However, NUC-suppressed HBV replication and diminished cccDNA transcription do not substantially lower circulating HBsAg levels in blood. This discrepancy is partly explained if S-HBs from iDNA is not affected by NUC therapy. We developed novel tools to quantify the contribution of iDNA vs. cccDNA transcription to surface (S) mRNAs and apply them to liver tissues from people with chronic HBV infection at various stages of hepatitis B. Unraveling the source of HBsAg in different disease states, which are on the spectrum leading towards HBsAg loss, will facilitate tailoring therapeutics that result in a functional cure for distinct individuals.

We have developed sensitive and specific molecular assays that characterize and quantify S transcripts derived from cccDNA vs iDNA that we have applied to liver tissues from people at different stages of HBV infection. We have used extant HBV transcriptional maps to profile mRNAs that derive from iDNA (3′ viral-human transition) vs. cccDNA (3′ viral). Using these S mRNA maps, we designed individual ddPCR primer/probes targeting the common S mRNA (mid-HBV) and the 3′-HBV ends of the S mRNAs to detect whether an S mRNA is intact (from cccDNA) or transitions to human sequence prior to the 3′ viral end (from iDNA). By multiplexing our optimal primer/probe sets, full-length S mRNAs (i.e., cccDNA-derived) fluoresce with the mid-HBV primers/probes (which is at the 5′ region of the S transcripts) and the 3′-HBV primers/probes; S mRNAs that have 3′ viral-human transitions from iDNA only fluoresce with the mid-HBV primer/probes. We have applied these probes and primers in quantitative PCR (qPCR) platform and ddPCR platform to cDNA from archived bulk liver tissues from different people and compared proportions of S mRNA derived from cccDNA and from iDNA between people over time. We found a tight correlation between the decline in HBsAg levels and whether S mRNAs were derived from cccDNA or iDNA, strongly indicating that our tool could be of major importance in stratifying people based on their predominant source of HBsAg.

Since NUCs continue to be a backbone of HBV treatment especially in PLWH, understanding the source of tHBsAg during NUCs is crucial for a functional cure. iDNA largely encodes for S-HBs while cccDNA encodes for L-, M-, and S-HBs: thus, we propose that if cccDNA transcription is diminished with NUCs (as we have shown), circulating L- and M- would decline out of proportion to S-HBs. We have been funded to use liver tissues from paired biopsies from HBeAg+ and HBeAg neg PLWH that have available contemporaneous serum. We apply the multiplex ddPCR to paired liver biopsies separated by 3-4 years to determine proportions of S mRNAs derived from cccDNA vs. iDNA. We expect ratios of cccDNA-derived S:iDNA-derived S mRNA to decrease in biopsy 2 compared to 1. Proportions of cccDNA-derived S:iDNA-derived S mRNA will be correlated with plasma L-, M-, S-HBs levels. This will allow us to calibrate our multiplex ddPCR assay to serum measurements that are typically obtained by clinicians.

To quantify the contribution of tHBsAg production from iDNA versus cccDNA in PLWH in different stages of hepatitis B infection. Hypothesis: Contribution to tHBsAg from cccDNA is greatest in untreated hepatitis B e antigen (HBeAg)+CHB and lowest in inactive CHB. We develop a sensitive ddPCR to characterize and quantify S mRNAs derived from cccDNA vs iDNA using archived liver tissues from PLWH at different stages of HBV infection: a) untreated immune active CHB (HBsAg+, HBV DNA>2000 IU/mL; n=5), b) NUC treated immune active CHB (HBsAg+, HBV DNA<200 IU/ml; n=55), c) inactive CHB (HBsAg+, HBV DNA<2000 IU/mL untreated; n=4), and d) occult hepatitis B (HBsAg below level of detection of commercial assays, HBV DNA<500 IU/ml; n=5). We will first perform capture-based RNAseq on six bulk liver tissues with immune active CHB to map mRNAs (S mRNA maps) that derive from iDNA (3′ truncated) vs. cccDNA (no 3′ truncations). Using S mRNA maps, we will design individual ddPCR primer/probes targeting the common 5′ (5′-S) and differing 3′ (3′-S) ends of the S mRNAs to detect whether an S mRNA is intact (from cccDNA) or truncated at the 3′ end (from iDNA). By multiplexing our optimal primer/probe sets, full-length S mRNAs (i.e., cccDNA-derived) fluoresce with 5′-S and 3′-S primer/probes; 3′ truncated S mRNAs from iDNA only fluoresce with the 5′-S primer/probes. We will apply multiplex ddPCR assays to cDNA from archived bulk liver tissues from our 4 groups and compare proportions of S mRNA derived from cccDNA and from iDNA between groups.

To determine if the source of tHBsAg shifts from cccDNA to iDNA during NUCs in PLWH who have HBeAg+ or HBeAg− CHB and to correlate these shifts with changes in circulating L-, M-, and S-HBs. Hypothesis: Proportion of S-HBs increase while the proportion of L- and M-HBs will decrease during NUCs. Since NUCs continue to be a backbone of HBV treatment especially in PLWH, understanding the source of tHBsAg during NUCs is crucial for a functional cure. iDNA largely encodes for S-HBs while cccDNA encodes for L-, M-, and S-HBs: thus, we propose that if cccDNA transcription is diminished with NUCs (as we have shown), circulating L- and M- would decline out of proportion to S-HBs. We will use liver tissues from paired biopsies from HBeAg+ and HBeAg neg PLWH that have contemporaneous serum (n=60). We will apply multiplex ddPCR (aim 1) to paired liver biopsies separated by 3-4 years (n=60, 120 biopsies) to determine proportions of S mRNAs derived from cccDNA vs. iDNA. We expect ratios of cccDNA-derived S:iDNA-derived S mRNA to decrease in biopsy 2 compared to 1. Proportions of cccDNA-derived S:iDNA-derived S mRNA will be correlated with plasma L-, M-, S-HBs levels. We will address the role of HIV on HBV by correlating CD4+ T cell counts before and during ART with ratios of cccDNA-derived S:iDNA-derived S mRNA. Our proposal is innovative and translational, and our results will inform about the contribution of tHBsAg production derived from HBV integrated sequences in CHB and support the design of HBV cure strategies.

To further illustrate, curing HBV is a public health priority especially in People Living With HIV (PLWH). Hepatitis B virus (HBV) infects 257 million people and leads to nearly 1 million annual deaths from liver disease and hepatocellular carcinoma. The WHO has called for global elimination of hepatitis B that includes treatment of 90% of those who need it. From a public health perspective, curative treatment is strongly preferred to sustaining lifelong treatment for such a large number of persons. The current HBV cure goal is functional cure, which is defined as loss of total hepatitis B surface antigen (tHBsAg). However, there are major gaps in our knowledge of how to achieve functional cure, with a major barrier being understanding the sources of tHBsAg in various HBV disease stages. Such knowledge is especially important in PLWH since HIV negatively affects CHB progression and tHBsAg loss. Up to 28% of PLWH also have CHB. Liver disease is one of the leading causes of morbidity and mortality in HIV-infected individuals. After acute HBV infection, HIV increases the risk of developing CHB. Further, HIV increases the risk for end stage liver disease and liver-related mortality from CHB. PLWH with lower CD4+ T cell nadir are less likely to clear tHBsAg during treatment with nucleos(t)ide inhibitors (NUCs) than those with higher counts. Moreover, in PLWH, tHBsAg titers are elevated with CHB compared to HBV mono-infected people and are also higher with lower CD4+ T cell counts. tHBsAg levels decreased more in co-infected people who experienced increases in CD4+ T cells with antiretroviral therapy (ART) than in those with smaller increases. Thus, understanding how HIV and CD4+ T cell loss are associated with tHBsAg production are important in developing an HBV cure for PLWH.

Various stages of HBV infection have been described. After acute infection, the immunotolerant stage is characterized by high HBV DNA levels, hepatitis B e antigen (HBeAg) positivity, and normal liver enzymes (ALT and AST). With triggering of an immune response, immune active chronic hepatitis B (CHB) begins with decline of HBV DNA, elevation in liver enzymes, and persistent HBeAg positivity. Some individuals will go on to develop inactive CHB, defined by an immune response that is robust enough to result in loss of HBeAg, undetectable levels of HBV DNA, and decline in HBsAg. From this stage, some individuals can lose HBsAg, develop anti-HBs antibodies, and recover from HBV (which resembles functional cure). However, others reactivate HBV with a mutated virus that does not produce HBeAg and leads to HBeAg negative immune active CHB with elevation in liver enzymes and a rise in HBsAg levels. Individuals do not need to progress through all stages and can also go back to an earlier stage. Lastly, some individuals have occult hepatitis B, defined as HBsAg levels that are below the limit of detection of commercial HBsAg assays, and low levels of HBV DNA. This scenario falls somewhere between inactive CHB and HBV recovery.

HBV enters the hepatocyte and is uncoated in the cytoplasm, exposing the relaxed circular HBV DNA (rcDNA). rcDNA traffics to the nucleus where host enzymes complete the partial double strand, converting it to cccDNA persists for the lifetime of the cell. cccDNA is the template for transcription of all five viral mRNAs and protein products. Specifically, the 2.4 kb mRNA yields the pre-S1+pre-S2+S (which is translated into the Large-HBsAg [L-HBs]), the 2.1 kb has different open reading frames that yield the pre-S2+S (Medium-HBsAg [M-HBs]) and the S (Small-HBsAg [S-HBs]), one 3.5 kb mRNA (also called preC) yields HBeAg, Pol, and core, a 0.7 kb mRNA yields X, and another 3.5 kb mRNA yields the pregenomic RNA (pgRNA) that is encapsidated and reverse transcribed by the viral polymerase (pol) to form additional rcDNA for progeny virions. In 10% of the time, an error in reverse transcription of pgRNA to rcDNA results in a double-stranded linear DNA (dsIDNA) molecule that can integrate into host genomic DNA and is referred to as integrated HBV DNA (iDNA). The major portion of the HBV genome that is integrated includes most of the surface (S) region, with several key features that distinguish it from S that derives from cccDNA: i) S integrands rarely contain pre-S1 or pre-S2 regions, and thus are likely to yield mostly S-HBs; ii) S integrands begin and terminate in human DNA; and iii) transition points between viral and human hybrid mRNAs that derive from S integrands are ‘truncated’ in their viral sequences at their 3′ end, whereas all mRNAs that derive from cccDNA terminate in a common 3′ region and poly-A tail.(13, 14).

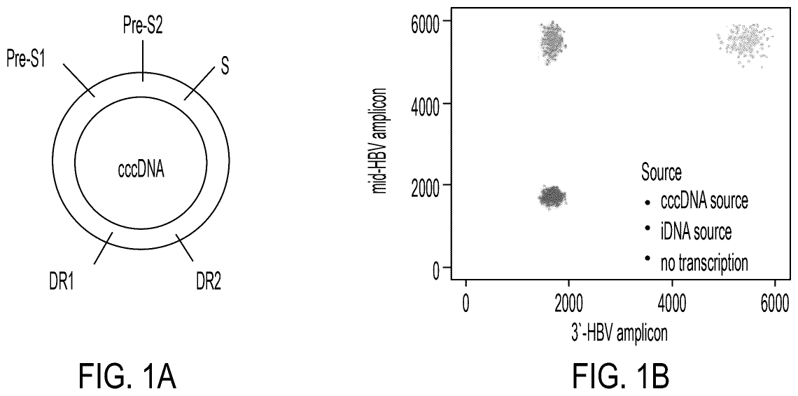

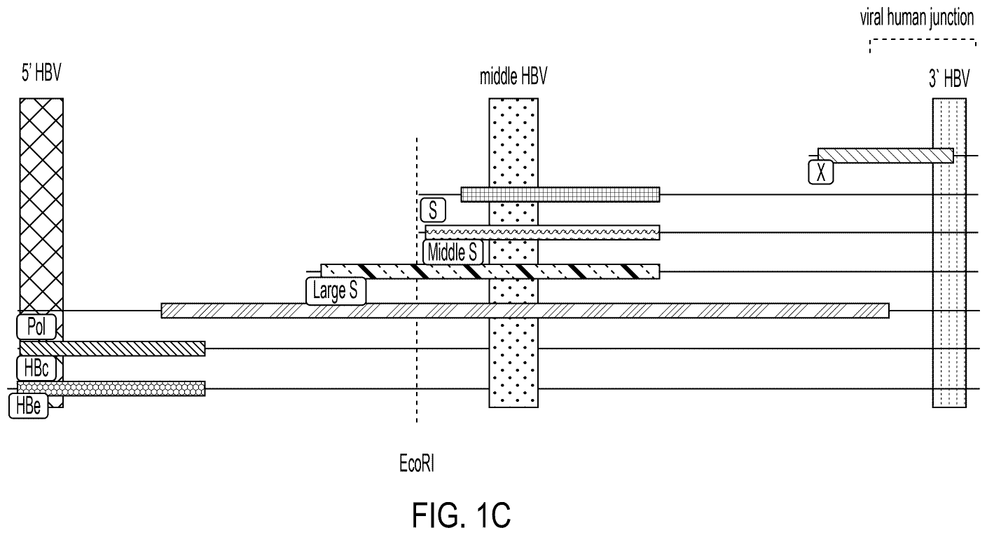

Among people who are infected, cccDNA is not readily eradicated despite antiviral therapy that suppresses plasma HBV DNA levels. Although the FDA-approved endpoint for functional cure is serum HBsAg loss, this measurement does not give a clear picture of the burden of cccDNA since HBsAg can also derive from iDNA (FIG. 1C). In addition, there is a discrepancy during antiviral therapy with nucleos(t)ide analogues (NUCs) between plasma HBV DNA and HBsAg levels: although plasma HBV DNA can be suppressed to below the limit of detection in people receiving NUCs, HBsAg remains detectable in the range of 10²-10⁶ IU/mL and does not decrease significantly during therapy. Transcription of iDNA could explain the discrepancy between HBsAg and HBV DNA levels during NUCs. Notably, S integrands produce HBsAg that derive from transcripts which typically have 3′ viral-human hybrid sequences.

Some embodiments utilize multiplex droplet digital polymerase chain reaction (ddPCR). This digital PCR technology separates nucleic acid templates into ˜20,000 nanoliter-sized droplets. A separate PCR amplification occurs within each droplet, allowing measurement of thousands of independent reactions. A specialized reader determines the proportion of PCR-positive droplets and uses Poisson statistics to quantitate template concentration in the original sample. We have validated our ddPCR to obtain sensitive quantitation of cccDNA and pgRNA from single hepatocytes. Our approach advances this technique by multiplexing ddPCR primers and probes to separately distinguish up to three different regions of the same strand of mRNA in the same reaction. By detecting the presence or absence of fluorescence of two separate non-overlapping amplicons, we can compute whether an S mRNA derived from iDNA or cccDNA.

Some embodiments involve linking liver tissue findings with characteristics of serum HBsAg and novel serum biomarkers of CHB. We use matched stored liver tissue and sera that are well-characterized for demographics. Thus, we are able to correlate the source of HBsAg with the size and abundance of L-, M-, S-HBs and quantitative (q)HBsAg in blood, which has not been done before.

Some embodiments involve, quantifying the contribution of HBsAg production from iDNA versus cccDNA for people with CHB in different stages of hepatitis B infection. There are two arms of an HBV cure: loss or silencing of cccDNA and loss of serum HBsAg. However, because HBsAg derives from cccDNA and iDNA, these two arms do not fully overlap. Since HBsAg is the clinical tool to determine whether an individual has achieved a cure, we developed molecular tools to quantify the separate contributions of iDNA and cccDNA transcription to S mRNAs and consequently to HBsAg production (which is the result of protein translation of S mRNAs) using biopsies from people with different stages of hepatitis B infection that produce different amounts of HBsAg.

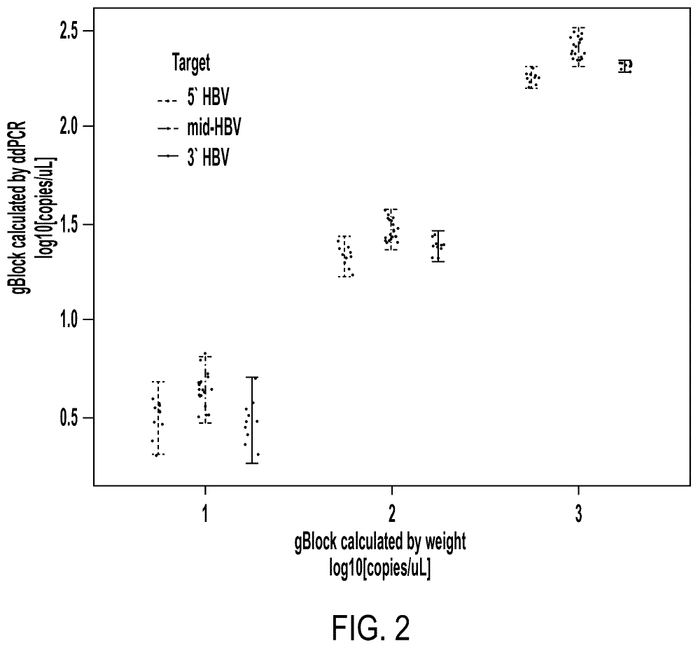

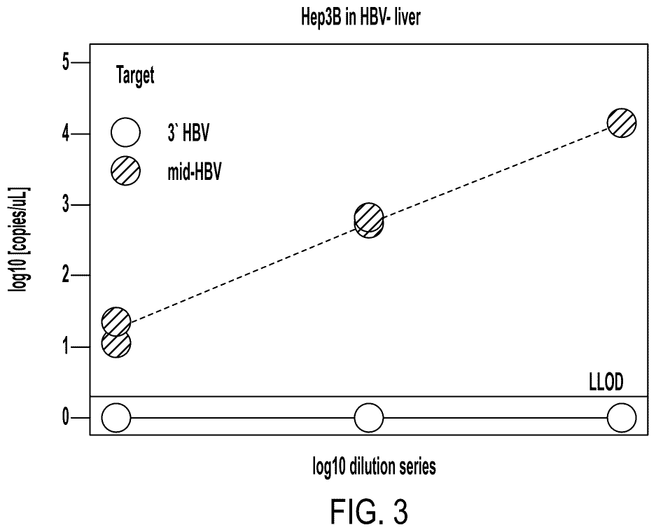

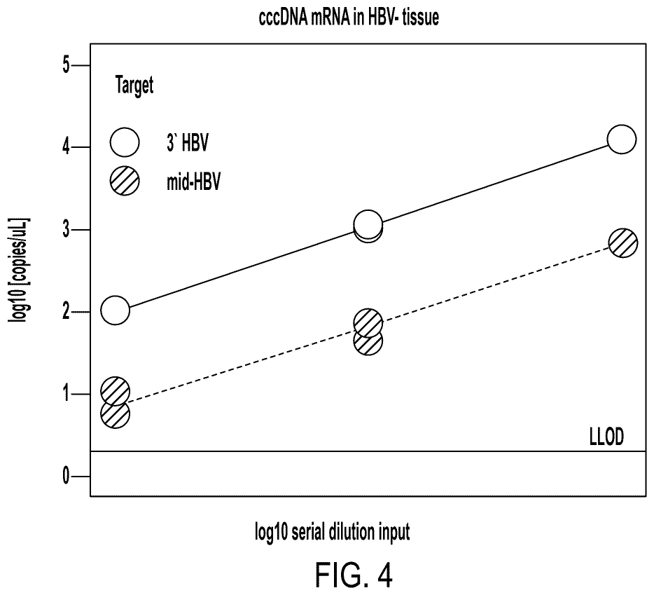

As described herein, we have demonstrated our facility in using high-resolution ddPCR tools to understand HBV transcription. Now, we have designed primers/probes that correspond to the mid-HBV region, which is synonymous with the 5′ region of S (downstream of pre-S1 and pre-S2) and separate primers/probes that correspond to the 3′-HBV, which is synonymous with the 3′ region of S that is also shared with all mRNAs that derive from cccDNA. When tested against a synthetic molecular standard in a multiplexed ddPCR assay, both sets of primers/probes demonstrate high sensitivity and specificity over a broad dynamic range (FIG. 2). When tested against HBV-negative tissue with a spike-in control of iDNA-derived RNA, the assay was shown to specifically quantify the mid-HBV region and none of the 3′-HBV region (FIG. 3). Furthermore, when applied to HBV-negative tissue with a spike-in control of cccDNA-derived RNA, the assay was shown to quantify both the mid-HBV and 3′-HBV regions as expected (FIG. 4).

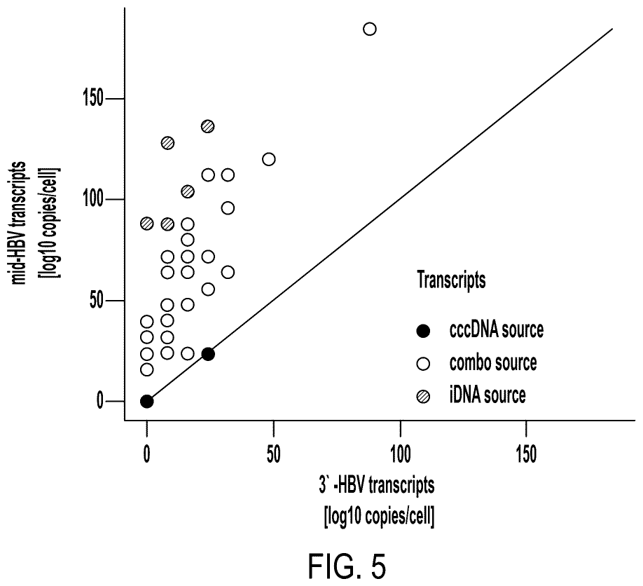

Some aspects involve single-cell analysis to determine heterogeneity of HBsAg production. An inherent limitation of bulk analyses is the inability to understand heterogeneity between cells as evidenced by our single-cell findings of HBV transcription exhibiting substantial variability between infected hepatocytes. Since some studies suggest that iDNA can be an infrequent event, we performed single cell analyses on liver tissue containing cells with all three classification types of HBV surface transcript sources: iDNA, cccDNA, combination of iDNA and cccDNA (FIG. 5). By accurately quantifying the presence or absence of mid-HBV and 3′-HBV amplicons in this multiplex ddPCR, we performed statistical analyses to define cells as sourcing surface transcripts wholly from cccDNA, partially from cccDNA and iDNA, or predominantly from iDNA. This mirrors our capability of classification in bulk tissue, but at a much more fine, granular level.

Some embodiments involve the goal of an HBV cure that has alternatively been stated to be eradication of cccDNA or HBsAg loss. However, because of the contribution of iDNA to HBsAg production, those goals are neither fully overlapping nor mutually exclusive. By developing a suite of tools to quantify iDNA and its contribution to HBsAg production, we facilitate design of novel therapies to target HBsAg based on its origin.

Some embodiments parlay the use of our single-cell laser capture microdissection (scLCM)/multiplexed ddPCR approach to intensively focus on single hepatocytes with iDNA-derived and cccDNA-derived S to confirm that expansion of hepatocytes with iDNA-derived S underlies persistent HBsAg levels.

### Exemplary Methods

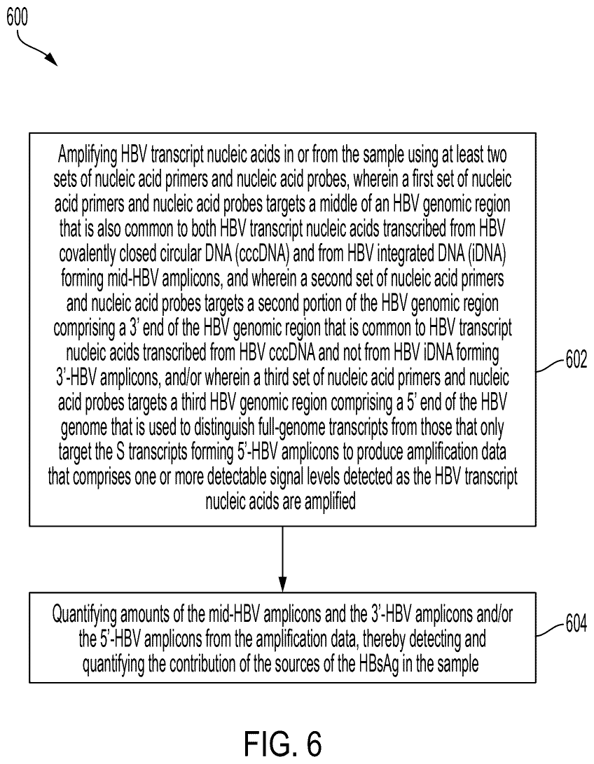

In some aspects, the present disclosure provides a method of detecting sources of hepatitis B virus (HBV) surface antigen (HBsAg) in a sample. To illustrate, FIG. 6 schematically shows method 600 that includes amplifying HBV transcript nucleic acids in or from the sample using at least two sets of nucleic acid primers and nucleic acid probes (e.g., in a multiplexed reaction mixture), wherein a first set of nucleic acid primers and nucleic acid probes targets a middle of an HBV genomic region that is also common to both HBV transcript nucleic acids transcribed from HBV covalently closed circular DNA (cccDNA) and from HBV integrated DNA (iDNA) forming mid-HBV amplicons, and wherein a second set of nucleic acid primers and nucleic acid probes targets a second portion of the HBV genomic region comprising a 3′ end of the HBV genomic region that is common to HBV transcript nucleic acids transcribed from HBV cccDNA and not from HBV iDNA forming 3′-HBV amplicons, and/or wherein a third set of nucleic acid primers and nucleic acid probes targets a third HBV genomic region comprising a 5′ end of the HBV genome that is used to distinguish full-genome transcripts from those that only target the S transcripts forming 5′-HBV amplicons to produce amplification data that comprises one or more detectable signal levels detected as the HBV transcript nucleic acids are amplified (step 602). Method 600 also includes quantifying amounts of the mid-HBV amplicons and the 3′-HBV amplicons and/or the 5′-HBV amplicons from the amplification data (step 604).

In some embodiments, the method further includes obtaining the sample from a subject. In some embodiments, the sample comprises hepatocellular tissue, serum, and/or plasma. In some embodiments, the methods include amplifying the HBV transcript nucleic acids in or from the sample using a digital droplet polymerase chain reaction (ddPCR) technique. In some embodiments, the method further comprises using the method to stratify subjects into different patient populations based upon the quantified amounts of the mid-HBV amplicons and the 3′-HBV amplicons and/or the 5′-HBV amplicons quantified for a given subject. In some embodiments, the method comprises administering one or more therapies to the subject based at least in part on a ratio of the first amount and the second amount. In some embodiments, the method comprises discontinuing administering one or more therapies to the subject based at least in part on a ratio of the first amount and the second amount. In some embodiments, the method further comprises correlating the quantified amounts of the mid-HBV amplicons and the 3′-HBV amplicons and/or the 5′-HBV amplicons with plasma levels of one or more of the HbsAgs in the subject. In some embodiments, the method further comprises quantifying the amounts of the mid-HBV amplicons and the 3′-HBV amplicons and/or the 5′-HBV amplicons from multiple samples obtained from the subject at different time points.

In some embodiments, the sets of nucleic acid primers and nucleic acid probes are selected from the group consisting of: a set of nucleic acids that targets a 5′ end of pre-genomic HBV RNA forming the 5′-HBV amplicons; a set of nucleic acids that targets all types of HBV transcript nucleic acids forming the mid-HBV amplicons; and a set of nucleic acids that targets a 3′ end of HBV transcript nucleic acids that are common to all HBV transcript nucleic acids that are transcribed from cccDNA forming the 3′-HBV amplicons. This group of nucleic acid primer/probes was matched to over 4,200 HBV sequences representing 9 genotypes to determine the applicability of the assay as a pangenotypic quantitative strategy (Table 3). The primers/probes bind with high efficiency across genotypes accounting for one nucleotide mismatch except for the midHBV probe in genotype B. In some embodiments, the sets of nucleic acid primers and nucleic acid probes are selected from the group consisting of: a set of nucleic acids having nucleotide sequences of SEQ ID Nos: 1, 2, and 3; a set of nucleic acids having nucleotide sequences of SEQ ID Nos: 4, 5, and 6; and a set of nucleic acids having nucleotide sequences of SEQ ID Nos: 7, 8, and 9 (Table 1). In some embodiments, the subject is co-infected with human immunodeficiency virus (HIV) and HBV and wherein a detected decline in total quantitative HBsAg (qHBsAg) of >0.5 log₁₀ international units per mL in the subject indicates that the HBV transcript nucleic acids are transcribed primarily from the HBV cccDNA. In some embodiments, the subject is co-infected with human immunodeficiency virus (HIV) and HBV and wherein an absence of a detected decline in total quantitative HBsAg (qHBsAg) in the subject indicates that the HBV transcript nucleic acids are transcribed primarily from the HBV iDNA.

TABLE 1

| Primer/ | | SEQ ID |
|---|---|---|
| Probe | Sequence | NO: |
| Forward | CTCGTGGTGGACTTCTCTC | 1 |
| Reverse | CAGCAGGATGAAGAGGAA | 2 |
| Probe | CCAACCTCCAATCACTCACCAACCT | 3 |
| Forward | CACCTCTGCCTAATCATC | 4 |
| Reverse | GGAAAGAAGTCAGAAGGCAA | 5 |
| Probe | TGTCCYACTGTTCAAGCCTCCAAGC | 6 |
| Forward | TAGGAGGCTGTAGGCATAA | 7 |
| Reverse | CACAGCTTGGAGGCTTGAA | 8 |
| Probe | TGCAACTTTTTCACCTCTGCCTAATCATCT | 9 |

TABLE 2

| Genotype | | | | | | | | | |
|---|---|---|---|---|---|---|---|---|---|
| (n | 5′-HBV | | | mid-HBV | | | 3′-HBV | | |
| sequences) | 4 | 6 | 5 | 1 | 3 | 2 | 7 | 9 | 8 |
| A | 99 | 99 | 95 | 99 | 100 | 100 | 99 | 99 | 100 |
| (508) | | | | | | | | | |
| B | 99 | 99 | 94 | 98 | 3 | 89 | 93 | 96 | 100 |
| (1000) | | | | | | | | | |
| C | 99 | 97 | 97 | 99 | 96 | 100 | 84 | 99 | 100 |
| (1543) | | | | | | | | | |
| D | 98 | 98 | 91 | 100 | 99 | 99 | 97 | 95 | 100 |
| (727) | | | | | | | | | |
| E | 100 | 97 | 96 | 100 | 98 | 100 | 98 | 98 | 99 |
| (230) | | | | | | | | | |
| F | 99 | 100 | 2 | 99 | 100 | 100 | 99 | 96 | 100 |
| (170) | | | | | | | | | |
| G | 100 | 95 | 0 | 100 | 0 | 100 | 100 | 60 | 95 |
| (20) | | | | | | | | | |
| H | 94 | 100 | 0 | 100 | 0 | 100 | 100 | 82 | 100 |
| (17) | | | | | | | | | |
| I | 100 | 97 | 93 | 100 | 97 | 100 | 100 | 100 | 100 |
| (29) | | | | | | | | | |

### Exemplary Systems and Computer Readable Media

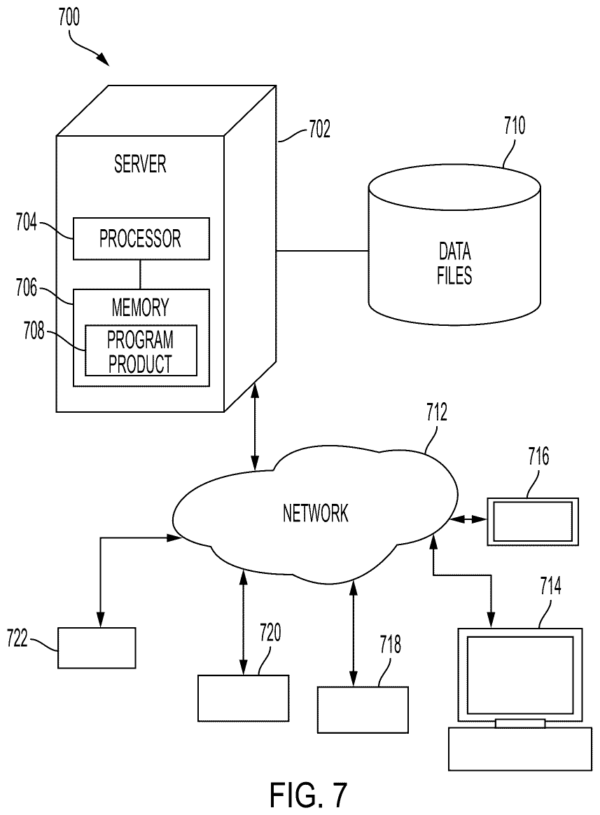

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 7 provides a schematic diagram of an exemplary system suitable for use with implementing at least aspects of the methods disclosed in this application. As shown, system 700 includes at least one controller or computer, e.g., server 702 (e.g., a search engine server), which includes processor 704 and memory, storage device, or memory component 406, and one or more other communication devices 714, 716, (e.g., client-side computer terminals, telephones, tablets, laptops, other mobile devices, etc. (e.g., for receiving captured images for further analysis, etc.)) positioned remote from nucleic acid amplification component (e.g., a thermocycler or the like) 718, sample preparation component 720, and material transfer component 722, and in communication with the remote server 702, through electronic communication network 712, such as the Internet or other internetwork. Communication devices 714, 716 typically include an electronic display (e.g., an internet enabled computer or the like) in communication with, e.g., server 702 computer over network 712 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 700 also includes program product 708 stored on a computer or machine readable medium, such as, for example, one or more of various types of memory, such as memory 706 of server 702, that is readable by the server 702, to facilitate, for example, a guided search application or other executable by one or more other communication devices, such as 714 (schematically shown as a desktop or personal computer). In some aspects, system 700 optionally also includes at least one database server, such as, for example, server 710 associated with an online website having data stored thereon (e.g., entries corresponding to more reference images, indexed therapies, etc.) searchable either directly or through search engine server 702. System 700 optionally also includes one or more other servers positioned remotely from server 702, each of which are optionally associated with one or more database servers 710 located remotely or located local to each of the other servers. The other servers can beneficially provide service to geographically remote users and enhance geographically distributed operations.

As understood by those of ordinary skill in the art, memory 706 of the server 702 optionally includes volatile and/or nonvolatile memory including, for example, RAM, ROM, and magnetic or optical disks, among others. It is also understood by those of ordinary skill in the art that although illustrated as a single server, the illustrated configuration of server 702 is given only by way of example and that other types of servers or computers configured according to various other methodologies or architectures can also be used. Server 702 shown schematically in FIG. 7, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 700. As also understood by those of ordinary skill in the art, other user communication devices 714, 716 in these aspects, for example, can be a laptop, desktop, tablet, personal digital assistant (PDA), cell phone, server, or other types of computers. As known and understood by those of ordinary skill in the art, network 712 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 708 is optionally in the form of microcode, programs, cloud computing format, routines, and/or symbolic languages that provide one or more sets of ordered operations that control the functioning of the hardware and direct its operation. Program product 708, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 708 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 708 is optionally copied from the computer-readable medium to a hard disk or a similar intermediate storage medium. When program product 708, or portions thereof, are to be run, it is optionally loaded from their distribution medium, their intermediate storage medium, or the like into the execution memory of one or more computers, configuring the computer(s) to act in accordance with the functionality or method of various aspects. All such operations are well known to those of ordinary skill in the art of, for example, computer systems.

To further illustrate, in certain aspects, this application provides systems that include one or more processors, and one or more memory components in communication with the processor. The memory component typically includes one or more instructions that, when executed, cause the processor to provide information that causes at least one captured tissue images and/or the like to be displayed (e.g., via communication devices 714, 716 or the like) and/or receive information from other system components and/or from a system user (e.g., via communication devices 714, 716, or the like).

In some aspects, program product 708 includes non-transitory computer-executable instructions which, when executed by electronic processor 704 perform at least: quantifying amounts of the mid-HBV amplicons and the 3′-HBV amplicons and/or the 5′-HBV amplicons from the amplification data to thereby detecting and quantifying the contribution of the sources of the HBsAg in the sample.

System 700 also typically includes additional system components (e.g., nucleic acid amplification component (e.g., a thermocycler or the like) 718, sample preparation component 720, and material transfer component 722) that are configured to perform various aspects of the methods described herein. In some of these aspects, one or more of these additional system components are positioned remote from and in communication with the remote server 702 through electronic communication network 712, whereas in other aspects, one or more of these additional system components are positioned local, and in communication with server 702 (i.e., in the absence of electronic communication network 712) or directly with, for example, desktop computer 714.

### EXAMPLES

### Example 1: Novel Multiplex Droplet Digital PCR Approach Reveals Dynamics of HbsAg Derived from Integrated HBV (iDNA)

### Background

Functional hepatitis B virus (HBV) cure is loss of circulating hepatitis B surface antigen (HbsAg). With current anti-HBV therapy, HBsAg usually persists, but it is unknown whether the source is either covalently closed circular DNA (cccDNA), iDNA, or both. To address this, we developed a novel approach using qPCR and multiplex droplet digital PCR (ddPCR) assays to discriminate iDNA-derived from cccDNA-derived surface transcripts(S) on 16 paired liver tissues.

### Methods

Our approach uses 3 distinct primer/probe pairs of comparable efficiency that target (A) 5′ end of pre-genomic RNA (5′-HBV amplicon), (B) all S and longer transcripts (mid-HBV amplicon), and (C) common 3′ end of all cccDNA-derived transcripts (3′-HBV amplicon). iDNA yields viral-human hybrid S transcripts that lack the viral downstream target (C). Quantities yielded by these assays were used to determine the relative amounts of iDNA-compared to cccDNA-derived S by estimating the amount of S transcripts that lacked common 3′ ends in RNA extracts from 16 paired liver tissues (7 HBeAg+ and 9 HBeAg− subjects). Biopsies were separated by 3-4 years on HBV-active antiretroviral therapy (ART). Total (unfractionated) quantitative HbsAg (qHBsAg) was measured by ELISA in contemporaneous serum samples.

### Results

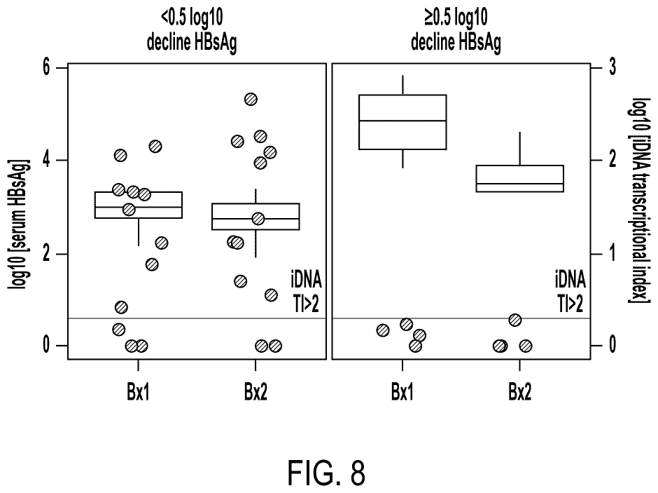

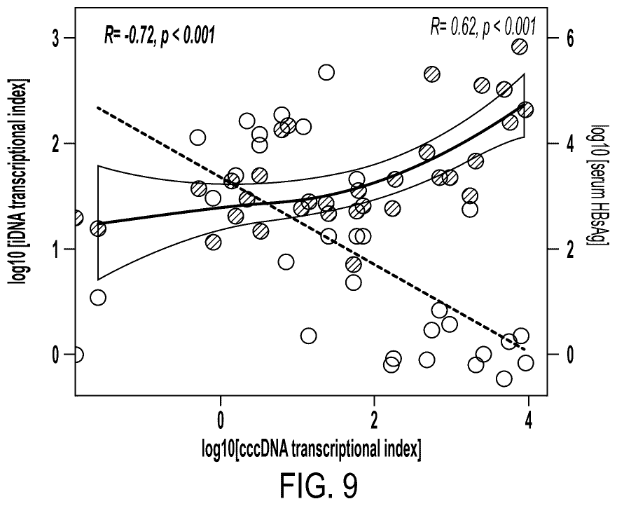

In participants with <0.5 log 10 decline HBsAg (range: −0.07-0.44), 3/12 were transcribing from predominantly cccDNA at biopsy 1 and 2/12 at biopsy 2. In participants with 0.5 log 10 decline HBsAg (range: 0.52-1.96), 4/4 were transcribing predominantly from cccDNA at both biopsies (FIG. 8). We further showed that as cccDNA is transcriptionally silenced in association with therapy, iDNA transcripts are enriched (FIG. 9).

### Example 2: Integrated Hepatitis B Virus DNA Maintains Surface Antigen Production During Antivirals

### Introduction

For the nearly 300 million people chronically infected with hepatitis B virus (HBV), current treatments, which include pegylated interferon-α (PEG-IFNα) and nucleos(t)ide analogues (NUCs), uncommonly result in cure, defined as a sustained loss of detectable HBV surface antigen (HBsAg) and HBV DNA in blood after cessation of therapy. NUCs inhibit HBV replication by interrupting HBV polymerase, a reverse transcriptase that converts viral pre-genomic RNA (pgRNA) to relaxed circular DNA (rcDNA), the infectious genome detected in plasma. Importantly, NUCs do not directly affect the covalently closed circular DNA (cccDNA) present in the nucleus of every infected cell, and thus cannot, on their own, result in HBV eradication. Furthermore, although NUCs are the mainstay of antiviral treatment in CHB, their suppressive effect on HBV viremia belies their limited impact on plasma HBsAg levels.

Although a central tenet of functional HBV cure is sustained loss of HBsAg in blood, it is important to acknowledge that HBsAg is a heterogeneous group of related proteins; transcription of distinct surface (S) mRNAs correspond to 2.4 kb (large ORF) and 2.1 kb (middle and small ORFs) transcripts. In addition, S mRNAs can be transcribed from either cccDNA or numerous integrated HBV DNAs (iDNA) found interspersed throughout the human genome. iDNAs are stable integrands of the truncated viral genome capable of producing complete S ORF transcripts in abundance. They can be distinguished from cccDNA-derived transcripts because iDNA-derived S mRNAs are chimeric with host DNA and lack the common 3′ ends that all cccDNA-derived transcripts share. Previously, we used single-cell laser capture microdissection (scLCM) to report that in persons taking long-standing NUCs, cccDNA transcription was markedly diminished and even silenced. While intriguing on its own, the observation raises the question as to why HBsAg levels did not decline commensurately during NUC treatment. We hypothesized that the continued production of HBsAg in persons taking NUCs is due to continued S mRNA transcription from iDNA.

To address this hypothesis, we developed a multiplex droplet digital PCR (ddPCR) assay that distinguishes between cccDNA- and iDNA-derived S transcripts. We applied our assay to paired bulk liver biopsies from individuals in a well-characterized HIV/HBV coinfection cohort who received NUCs and had longitudinal follow-up. We linked hepatic measurements with peripheral quantities of HBsAg. In a subset of these paired biopsies, we combined scLCM and multiplex ddPCR to compare levels of cccDNA-derived and iDNA-derived S transcripts in individual hepatocytes.

### Results

A Multiplex ddPCR Assay for iDNA-Derived Transcripts

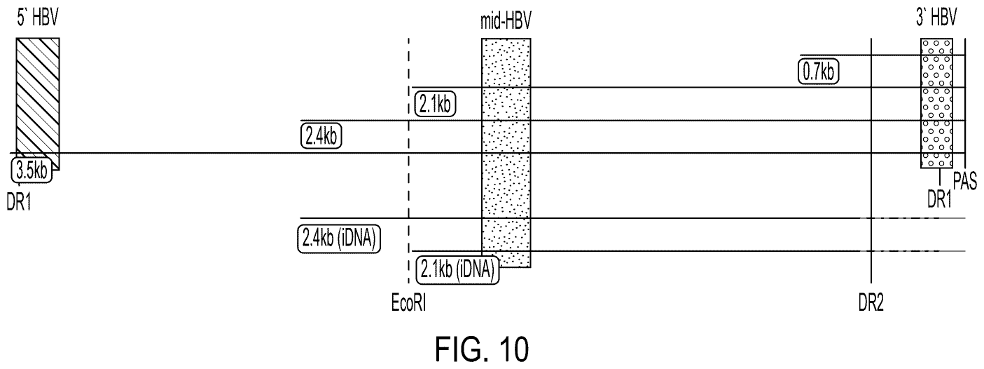

We developed a discriminatory assay exploiting the common cccDNA-derived mRNA 3′ overlaps that terminate in a common poly-A signal (PAS). Previously published long- and short-read sequencing transcriptome maps of HBV-infected liver tissues indicate that iDNA-derived transcripts, overwhelmingly S mRNAs, lack the common PAS. Thus, we designed a multiplex ddPCR assay to independently quantify the middle (mid-HBV) and 3′ (3′ HBV) ends of HBV mRNAs to distinguish transcripts that derive from cccDNA from those that derive from iDNA. The mid-HBV assay was designed to align against all S mRNAs as well as 3.5 kb mRNA if present. We added a 5′ assay (5′ HBV) quantifying pgRNA as an independent measure of transcriptional activity from cccDNA since transcriptome maps rarely demonstrate iDNA-derived pgRNA (FIG. 10). Using these multiple ddPCR assays in combination, iDNA-derived transcripts can be distinguished from cccDNA-derived transcripts when the mid-HBV region is amplified but the 3′ HBV assay is not. We verified the specificity of this approach by applying these assays to transcripts taken from Hep3B cells, which only contain iDNA, that were added to HBV-uninfected liver tissue and diluted. We confirmed that only the mid-HBV amplicon, and not the 3′ HBV amplicon, was quantifiable over a dynamic range. Thus, we concluded this approach was specific for iDNA-derived transcripts. We applied the same multiplex assay to liver from a mouse model of HBV infection (see Methods). C57BL/6 mice were transfected with a cccDNA expression vector from which all canonical HBV transcripts are produced. We applied the mid-HBV and 3′ HBV assays to homogenized liver extracts from these mice and found that both assays quantified abundant transcripts over a dynamic range, confirming their sensitivity. In addition, we tested each assay individually and in combination against purified synthetic DNA standards in equimolar concentrations across a range of dilutions and found comparable PCR efficiencies. The lower limit of detection (LLOD) for each amplicon was <10 copies/μL input. Finally, we verified in silico that these primers would work across genotypes by demonstrating that our primers and probes bind equally to more than 4,000 full length HBV sequences of varying genotypes.

Multiplex ddPCR Assay Correlates with HBsAg Levels in Blood

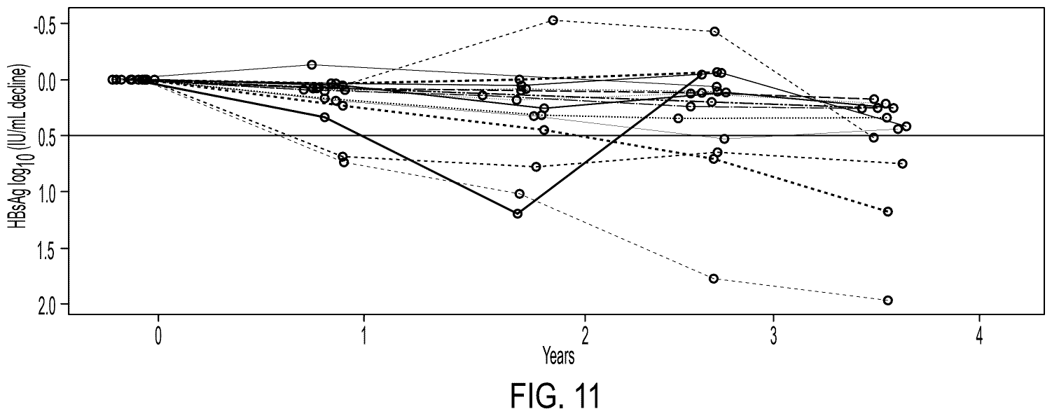

We applied the multiplex ddPCR assay to paired liver biopsies from 16 HIV/HBV coinfected persons enrolled in the Hepatitis B Research Network Ancillary Study (16) at Johns Hopkins Hospital (Table 3). Each participant had fresh frozen paired liver biopsies separated by a median of 3.5 years (range: 2.7-3.8 years). At biopsy 1, 12/16 people were on NUC therapy for more than one year and all were on therapy between biopsies. All but two people at biopsy 1 and one at biopsy 2 had CD4+ T cell counts>200 cells/mm³ (median 558 cells/mm³ and 659 cells/mm³ at biopsies 1 and 2, respectively). HIV RNA was undetectable in 13/16 individuals at each biopsy. At biopsy 2, the maximum HIV RNA was 1.7 log₁₀ copies/ml. HBV DNA was detectable in 8/16 and 6/16 people at biopsy 1 and 2, respectively, and the median HBV DNA decline between biopsies was 1.25 log₁₀ IU/mL (IQR: −0.3-6.3 log₁₀). None of the participants had HBsAg loss during treatment. The median HBsAg levels at biopsies 1 and 2 were similar (3.2 log₁₀ (IQR: 3.0-3.6 log₁₀) IU/mL and 2.9 log₁₀ (2.8-3.3 log₁₀) IU/mL, respectively, p=0.3). Overall, the median HBsAg level declined minimally between biopsies by a median 0.26 log₁₀ IU/mL (IQR: 0.15-0.48 log₁₀ IU/mL decline). Notably, four (25%) people achieved>0.5 log₁₀ IU/mL HBsAg decline (range: 0.52-1.96 log₁₀) whereas the median decline in the other 12 people was 0.23 log₁₀ IU/mL (IQR: 0.09-0.3 log₁₀; FIG. 11). We stratified participants by the magnitude of their decrease in HBsAg quantities, comparing results of the four people in the highest quartile (>0.5 log IU/ml decline) to the remaining 12 individuals to test whether the magnitude of HBsAg decline during antiviral treatment could be attributed to the amount of iDNA-derived S mRNA.

TABLE 3
Study participant demographics and characteristics.

| | Biopsy 1 | Biopsy 2 |
|---|---|---|
| Age at Biopsy 1 median years | 53 [28,67] | |
| [min, max] | | |
| Race | | |
| African American | 14/16 | |
| Caucasian | 1/16 | |
| Other | 1/16 | |
| Male sex at birth | 16/16 | |
| Years of ART at baseline | 6.5 [0.1, 14.2] | |
| [min, max] | | |
| HBeAg positive^{A} | 9/16 | 8/16 |
| Median HBV DNA log₁₀ IU/mL | 1.0 [UND, 8.8] | UND [UND, 3.0] |
| [min, max] | | |
| Median HBsAg log₁₀ IU/mL | 3.2 [2.1, 5.8] | 2.9 [1.7, 5.1] |
| [min, max] | | |
| Median HIV RNA log₁₀ copies/mL | UND [UND, 4.7] | UND [UND, 1.7] |
| [min, max] ^{B} | | |
| Median CD4+ T cells/mm³ | 558 [153, 909] | 659 [139, 974] |
| [min, max]^{C} | | |

^{A}One study participant became HBeAg-negative <6 months after biopsy 1.
^{B} HIV RNA measurements within 6 months of biopsy were used, and when unavailable, the most recent HIV RNA measurement was used provided that HIV RNA levels had been stable. HIV RNA levels were unavailable for two people.
^{C}CD4+ T cell counts within 6 months of biopsy were used, and when unavailable, the most recent CD4+ T cell count was used, provided that CD4+ T cell counts had been stable. CD4+ T cell counts were unavailable for two people.
UND - undetectable

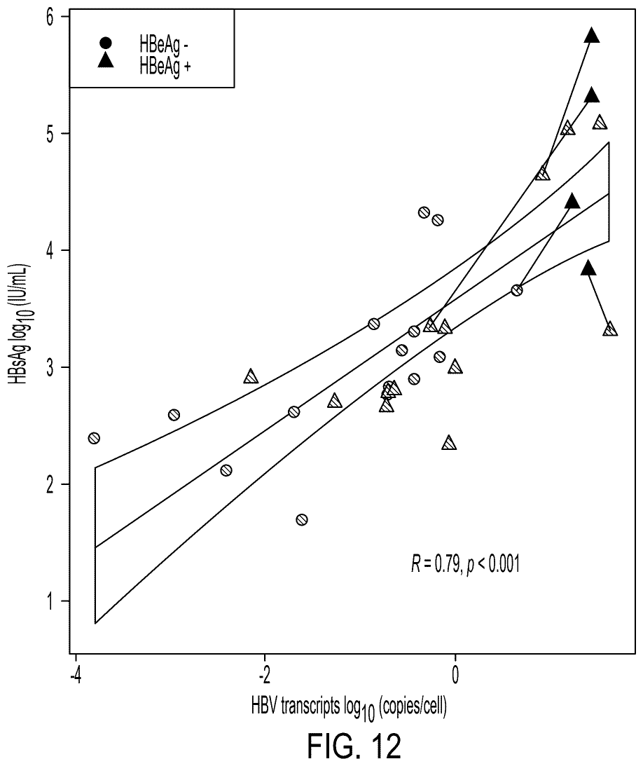

First, we confirmed that quantifying total HBV transcription, whether from cccDNA or iDNA, was an accurate correlate of HBsAg production. We applied the assay to RNA extracted from bulk liver tissue from the 32 (16 pairs) liver biopsies. We simultaneously quantified the total number of cells in each sample using a separate qPCR assay measuring ERV3 DNA, an endogenous retroviral sequence in every human cell. Because the mid-HBV assay measures all viral transcripts from cccDNA and iDNA except for the low abundance 0.7 kb HBX gene product (see Methods), we used this assay to estimate total HBV transcriptional activity. Adjusting to ERV3 quantities, HBV transcription/cell correlated closely with HBsAg levels (r=0.79; p<0.001; FIG. 12). This result was expected, since the mid-HBV assay targets an amplicon embedded in all S mRNAs, irrespective of their origin. Thus, we concluded that the mid-HBV assay is an adequate surrogate for S mRNAs that are translated into HBsAg, and that we could use the assay multiplexed with the 3′ HBV assays to unravel the source of HBsAg production. We also considered how HBV transcription varied with hepatitis B e antigen (HBeAg) status: HBsAg levels were marginally elevated in persons who were HBeAg-positive (median 3.33 log₁₀ IU/mL) compared to HBeAg-negative (2.90 log₁₀ IU/mL; p=0.069). Correspondingly, biopsies from HBeAg-positive individuals had higher levels of HBV transcription, quantified by the mid-HBV assay, compared to biopsies from HBeAg-negative individuals (p<0.01).

iDNA Transcription in Liver Explains the Response of Serum HBsAg Levels to NUCs

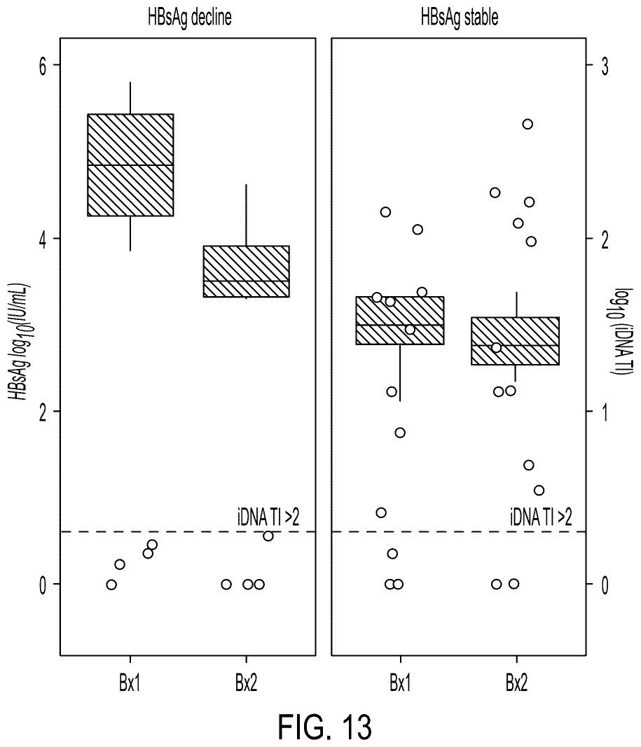

We next tested whether differences in iDNA transcription could explain the differences in decline in serum HBsAg between the four persons in the highest quartile who had a >0.5 log₁₀ decline during NUC therapy compared to the twelve others who did not. We calculated an iDNA transcriptional index (iDNA TI, see methods) defined as the ratio of cccDNA and iDNA-derived transcripts (mid-HBV) to only cccDNA-derived transcripts (3′ HBV amplicon). Thus, the iDNA TI represents a relative relationship between the quantity of iDNA- to cccDNA-derived S transcripts, facilitating comparison of the dominant source of S transcripts between biopsies regardless of the absolute quantity of S transcripts. An iDNA TI≤1 is interpreted as transcription only from cccDNA whereas an iDNA TI>2 represents HBsAg transcripts deriving predominantly from iDNA rather than from cccDNA. An iDNA TI between 1 and 2 represents contributions from iDNA and cccDNA without dominance of either. Interestingly, we found that individuals with 50.5 log₁₀ decline in HBsAg were transcribing predominantly from iDNA at both biopsies 1 and 2 with median iDNA transcriptional indices of 21.5 (IQR: 2.0-47.1) and 18.3 (IQR: 4.2-141.8), respectively (FIG. 13). However, in people who achieved >0.5 log₁₀ decline in HBsAg while on NUC therapy, viral transcription was primarily from cccDNA at biopsies 1 and 2 with median iDNA transcriptional indices of 1.4 (range: 0.9-1.7) and 0.9 (range: 0.9-1.9), respectively (FIG. 13). This difference in iDNA TI was statistically significant between those that achieved a >0.5 log₁₀ decline and those that did not (p<0.01). We confirmed that the total amount of serum HBsAg at either time point did not confound the relationship between the decline in HBsAg and the iDNA TI.

iDNA-Derived Transcription is a Dominant Source of S mRNAs During NUC Therapy

Because iDNA and cccDNA transcription can independently contribute to HBsAg, and because we previously reported that cccDNA transcription is silenced during NUCs, we next examined the relationship between iDNA and cccDNA transcription in the context of NUC therapy. We have previously used the 5′ HBV amplicon to quantify 3.5 kb transcripts with a particular interest in pgRNA. As these transcripts are almost exclusively a result of cccDNA transcription, we use this as a surrogate for cccDNA transcriptional activity, but conservatively designate the ratio of pgRNA:cccDNA molecules as the pgRNA transcriptional index (pgRNA TI; see Methods).

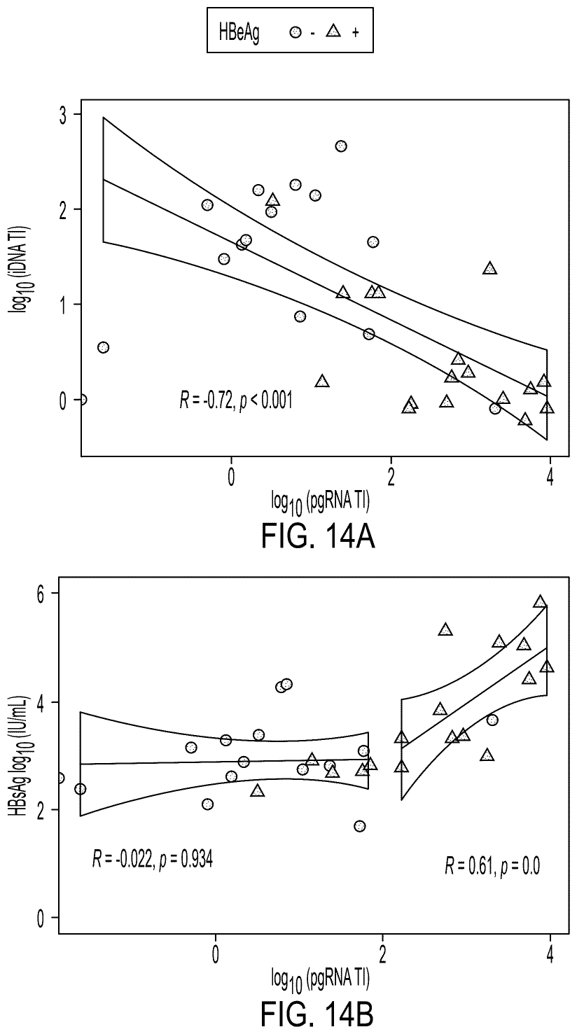

As in our prior single-cell studies, we found that NUC therapy duration was associated with reductions in cccDNA transcription (pgRNA TI) in bulk tissues (r=−0.51, p<0.01). Interestingly, while transcription from cccDNA during NUCs diminished, the relative transcription from iDNA increased: we observed that the pgRNA TI and the iDNA TI were inversely correlated (r=−0.72, p<0.001; FIG. 14A). This finding suggests that cccDNA transcription contributed less to S mRNAs during NUC therapy as iDNA transcription produced an increasing fraction of S mRNAs. This transcriptional switching parallels HBeAg status: HBeAg-negative persons were pgRNA TI^{low}/iDNA TI^{high} and HBeAg-positive persons were pgRNA TI^{high}/iDNA TI^{low} (iDNA TI p<0.01, pgRNA TI p<0.001).

To test whether cccDNA transcriptional silencing could be independently linked to HBsAg declines, we plotted the pgRNA TI against serum HBsAg (FIG. 14B). Intriguingly, we found that decline from high to low levels of cccDNA transcription (pgRNA TI) correlated with a decline in serum HBsAg levels to a threshold pgRNA TI of ˜100 (r=0.61, p<0.03). Below that threshold, serum HBsAg levels did not appear to decline any further (r=−0.022, p=0.9).

Single-Cell Viral Transcriptional Landscapes Reveal the Contributions of iDNA Versus cccDNA

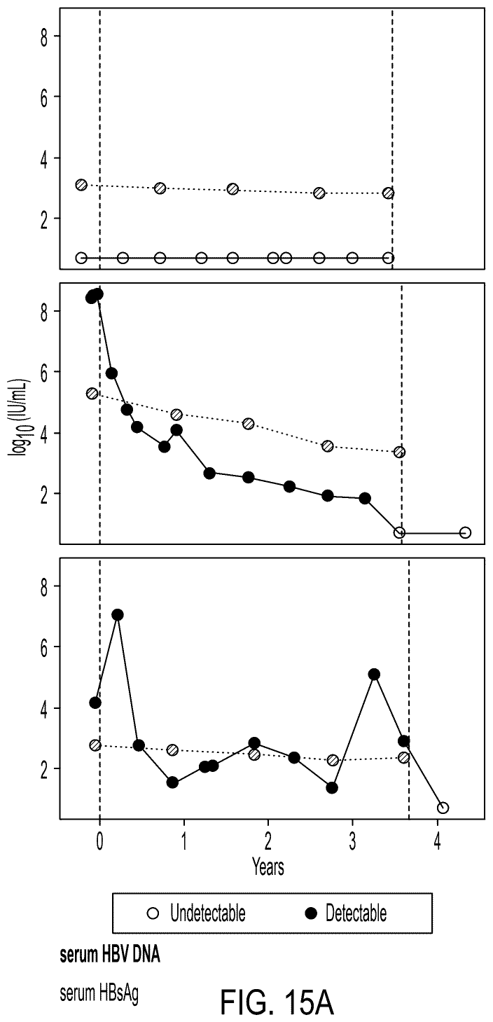

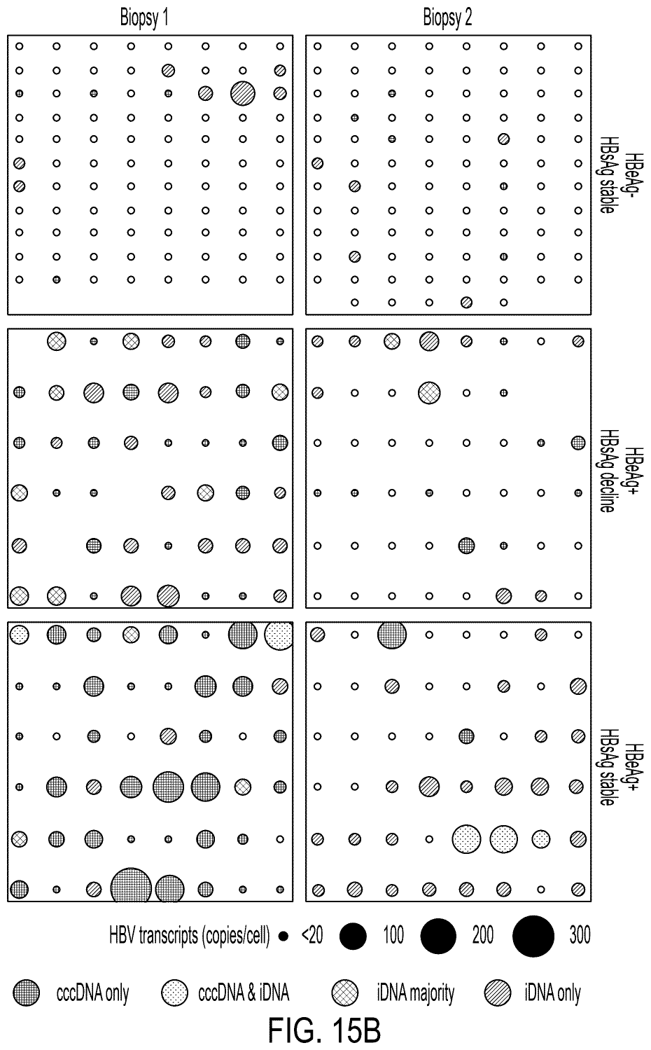

To further understand how iDNA maintains levels of HBsAg in the liver during NUC therapy, we examined individual hepatocytes from three individuals. We performed scLCM on paired biopsies from three of the 16 study participants who were representative of others in the cohort (Table 4): i) an individual who was HBeAg-negative and experienced minimal HBsAg decline; ii) an individual who was HBeAg-positive and experienced 1.96 log₁₀ IU/mL HBsAg decline; and iii) an individual who was HBeAg-positive and experienced minimal HBsAg decline. We included an analysis of the participants' serum HBV DNA that were available through the cohort, as a surrogate for the presence and efficacy of NUC-suppression of viral replication. We studied an array of hepatocytes, spatially consecutive as we have done previously, from each biopsy for the three persons. We applied multiplex ddPCR using the 5′, mid-, and 3′ HBV amplicons to the RNA extractions from each cell. We developed an algorithm using the iDNA TI to classify each cell by the relative contribution of iDNA transcription to S mRNAs: i) transcribing from cccDNA only (iDNA TI≤1); ii) transcribing from cccDNA and iDNA (1<iDNA TI≤2); iii) transcribing predominantly from iDNA (iDNA TI>2); and iv) transcribing only from iDNA (mid-HBV⁺/3′ HBV⁻; FIG. 15B). Thus, after excluding cells with incomplete measurements (26/384; 6.7%), the remaining cells could be indexed for transcriptional activity and the relative contributions from cccDNA or iDNA.

TABLE 4
Characteristics of select persons examined by scLCM

| | HBeAg+ | | HBeAg+ | | HBeAg− | |
|---|---|---|---|---|---|---|
| | HBsAg stable | | HBsAg decline | | HBsAg stable | |
| | Bx 1 | Bx 2 | Bx 1 | Bx 2 | Bx 1 | Bx 2 |
| HBsAg | 2.77 | 2.33 | 5.30 | 3.34 | 3.09 | 2.83 |
| log₁₀ | | | | | | |
| IU/mL | | | | | | |
| iDNA/cccDNA | 0.8 | 121.6 | 1.7 | 1.9 | 45.6 | 462.9 |
| transcripts | | | | | | |
| (iDNA TI; in | | | | | | |
| bulk tissue) | | | | | | |
| pgRNA TI | 165 | 3 | 565 | 938 | 60 | 24 |
| (in bulk | | | | | | |
| tissue) | | | | | | |
| serum HBV | 4.1 | 2.9 | 8.5 | UND | UND | UND |
| DNA log₁₀ | | | | | | |
| IU/mL | | | | | | |
| cccDNA/1000 | 4.3 | 1.1 | 75.3 | UND | 1.0 | 0.2 |
| cells | | | | | | |

iDNA TI—iDNA transcriptional index;
UND—undetectable;
HBeAg+—hepatitis B e antigen positive;
HBeAg−—hepatitis B e antigen negative

We designated a cell as transcriptionally active if either the 5′, mid-, or 3′ HBV amplicons were detectable. In the two HBeAg-positive individuals, we observed that some cells transcribed S more from iDNA relative to cccDNA whereas others transcribed more from cccDNA (FIG. 15B). In contrast, in the HBeAg-negative individual, all cells with S mRNAs transcribed chiefly from iDNA.

In the HBeAg-negative individual with stable HBsAg levels and undetectable HBV DNA at both biopsies (FIG. 15A), the proportion of transcriptionally active cells between biopsies was stable: 11/83 (13%) cells at biopsy 1 and 10/90 (11%) cells at biopsy 2. Of these transcriptionally active cells, 63% and 50% at Biopsy 1 and 2, respectively, transcribed predominantly from iDNA relative to cccDNA (FIG. 15B), although this difference was not statistically significant. Compared to bulk, there was a lack of enrichment of iDNA-derived transcripts between biopsies in this individual, although the single-cell work was not powered to estimate precise changes in iDNA transcription enrichment. In the HBeAg-positive individual with 1.96 log₁₀ IU/mL HBsAg decline between biopsies, the proportion of transcriptionally active cells decreased between the biopsies from 45/45 (100%) cells at biopsy 1 to 22/46 (48%) cells at biopsy 2 (FIG. 6B; p<0.001). This decrease in cellular transcriptional activity paralleled the declines in serum HBV DNA (FIG. 15A). Notably, the proportion of transcriptionally active cells that predominantly derived transcripts from iDNA was similar at biopsy 1 and 2 (56% and 45%, respectively). Further, the pgRNA TI from bulk tissue did not decline suggesting that hepatocytes that remained infected were transcribing from cccDNA at a steady rate. However, we did observe a considerable loss of detectable cccDNA in bulk tissues (Table 4). The decline in HBsAg in this HBeAg-positive individual, therefore, is best explained by a loss of infected hepatocytes, especially those that were transcribing primarily from cccDNA, rather than a change in transcription from either iDNA or cccDNA. This is in stark contrast to the HBeAg-positive individual with stable HBsAg between biopsies (FIG. 15A). While similar to the HBeAg-positive person with a significant HBsAg decline in that more cells at biopsy 1 than 2 were transcriptionally active (biopsy 1-44/48 [92%] cells; biopsy 2-29/46 [63%] cells; FIG. 15B), the fraction of transcriptionally active cells that derived S mRNAs from iDNA was enriched over time: from 7/44 (16%) cells at biopsy 1 to 24/29 (83%) at biopsy 2. This finding aligns closely with what we found in bulk tissue, where the iDNA TI increased from 0.8 to 121.6 (Table 4). Thus, we hypothesize that in this individual, despite loss of infected hepatocytes, there was enrichment in the proportion of cells transcribing from iDNA, which ultimately led to stable levels of serum HBsAg. Taken together, we found that while NUC-associated cccDNA transcriptional silencing is occurring, HBsAg levels appeared to be governed by whether and to what extent iDNA-transcription occurred.

### DISCUSSION

We developed a multiplex ddPCR assay to distinguish cccDNA- from iDNA-derived S transcripts and applied it to both bulk liver tissue and individual hepatocytes in paired liver biopsies from 16 individuals during 2-4 years of NUC therapy as a part of highly active antiretroviral therapy. We found that individuals who are HBeAg positive have greater relative S transcription from cccDNA whereas those who are HBeAg negative transcribe S predominantly from iDNA. Interestingly, for individuals in whom iDNA was the primary source of S transcripts at baseline, NUC treatment did not substantially reduce the levels of HBsAg in blood. In contrast, NUC treatment did reduce the levels of HBsAg in blood in individuals in whom cccDNA was the predominant source at baseline and we attribute this to cccDNA transcriptional silencing and clearance of infected cells. Our single cell data largely corroborate these findings but also allow a more granular view of how relative changes in iDNA versus cccDNA transcription within single hepatocytes relate to changes in total HBV transcription. Overall, our results highlight the importance of iDNA transcription in some individuals to sustain elevated HBsAg levels despite NUC treatment of CHB: in those individuals, the relative amount of iDNA- to cccDNA-derived transcription appeared to enrich over time. Thus, transcriptionally active iDNA contributes to circulating HBsAg during NUCs and is therefore a barrier to functional cure.

We clearly demonstrate that HBeAg-positive individuals transcribe S mostly from cccDNA whereas HBeAg-negative individuals transcribe S from iDNA predominantly. This difference in the origin of S transcripts was highlighted by Wooddell et al. when treating HBeAg-positive and HBeAg-negative chimpanzees, after NUC lead-in, with an siRNA targeting the shared 3′ end of cccDNA- but not iDNA-derived viral transcripts (Rnai-based treatment of chronically infected patients and chimpanzees reveals that integrated hepatitis b virus DNA is a source of hbsag, Sci. Transl. Med. 9, 1-12 (2017)). HBeAg-positive chimpanzees had significant HBsAg declines and robust cccDNA transcription in contrast to HBeAg-negative chimpanzees with stable HBsAg levels and predominantly iDNA transcription. After adjusting the siRNA target to silence both cccDNA- and iDNA-derived transcripts, HBsAg levels declined universally, highlighting the contribution of iDNA-derived S mRNAs to HBsAg production. Long read sequencing of RNA from two chimpanzees in this study showed that 77% of transcripts derived from iDNA in the HBeAg-negative chimpanzee compared to 10% from iDNA in the HBeAg-positive chimpanzee. Two other groups have reported a similar phenomenon in the context of hepatocellular carcinoma (HCC) as liver from people with HCC had 80-90% of viral transcripts deriving from iDNA rather than cccDNA though HBeAg status was not available for these studies. Although HBsAg levels in our 16 study participants were higher in HBeAg-positive individuals than in HBeAg-negative individuals, the difference was only borderline significant, highlighting that iDNA transcription in HBeAg-negative individuals can lead to high levels of HBsAg. There is also heterogeneity between individuals in the relative transcription from iDNA. This may be determined during the initial infection since increased initial viremia is associated with increased viral integration and prolonged HBsAg levels. It is intriguing to consider that earlier treatment may limit integration events and allow for more effective control of HBsAg production with NUCs.

We and others have reported that NUC therapy is associated with cccDNA transcriptional silencing, which can also be inferred from the decline of HBV RNA in serum as well as pgRNA in the liver. By comparing quantities of pgRNA to cccDNA in people taking NUCs who had decreases in serum HBV DNA, here we confirmed our earlier findings that NUCs are associated with transcriptional silencing of cccDNA, rather than the alternative hypothesis that HBV transcription is reduced during NUCs strictly because of an overall reduction in the number of infected cells. cccDNA transcriptional silencing during NUCs is likely to be transient, since treatment interruption is frequently associated with virologic rebound. After HBV DNA is no longer detectable in serum and HBV RNA has declined, substantial HBsAg quantities remain in the majority of NUC-treated persons, even if diminished. Our data offer a potential explanation for this previously unexplained observation in that the individuals who transcribed S mostly from cccDNA had significant HBsAg declines coupled with decreases in cccDNA transcription on NUC therapy whereas those who transcribed mostly from iDNA did not have substantial declines in HBsAg levels. These findings suggest that NUC therapy may modulate HBsAg levels in cccDNA transcriptionally dominant individuals but not in iDNA transcriptionally dominant individuals. A recent study by van Buuren et al. (Targeted long-read sequencing reveals clonally expanded HBV-associated chromosomal translocations in patients with chronic hepatitis B, JHEP Reports 4, 100449 (2022)) showed a similar phenomenon in NUC+PEG-IFNα treated individuals: iDNA-derived transcription was enriched when HBsAg levels remained stable, although the group was unable to determine whether this was an effect of PEG-IFNα or NUCs since all participants received PEG-IFNα. We now demonstrate enrichment of iDNA transcription relative to cccDNA transcription with NUCs, especially when cccDNA-derived transcription is progressively diminished. These observations may impact treatment strategies for people with CHB as currently NUC therapy is required lifelong to suppress HBV DNA and HBV RNA levels in serum. Our findings establish that the subset of people with HBsAg deriving mainly from iDNA may never achieve further declines in HBsAg from prolonged NUC therapy alone since NUCs are not known to inhibit transcription from iDNA, although they likely interrupt new integration events. We predict that the subset of individuals with primarily iDNA-derived HBsAg will require novel therapies targeting iDNA-derived S mRNAs or the cells that produce them.

Our single cell analysis highlights that HBV transcription is not static in the liver even though it may appear so when strictly looking at HBsAg levels. In the HBeAg-positive individual with stable HBsAg levels between biopsies (FIG. 15) the production of S transcripts switched from cccDNA to iDNA dominant. Whether this is due to prolonged NUC treatment or changes in host factors deserves further exploration. The single cell work also supports the findings that HBsAg loss is mainly due to diminished cccDNA transcription, possible cccDNA degradation, or loss of cells containing cccDNA, as evidenced by the HBeAg positive individual who had a decline in HBsAg coupled with a loss of detectable cccDNA in bulk. A conjecture that arises from the single-cell work is whether enrichment of iDNA transcriptionally active cells is indicative of clonal proliferation, if indeed NUCs completely suppress viral replication, which remains an open question. Each of the individuals selected for single cell work had different HBsAg trajectories or a different HBeAg status; thus, further single cell work is needed to determine whether there are other patterns of changes in individual hepatocytes over time with NUC therapy.

Current methods to determine the origin of a viral transcript as cccDNA or iDNA are cumbersome, expensive, and have a high degree of quantification error as they rely on Alu-PCR, RNA-seq, or sequential PCR and subtraction strategies. Our assay is not only broadly applicable for multiple genotypes, but also multiplexed so that quantification of total HBV transcripts and cccDNA-derived transcripts happens within the same reaction, thus nullifying the need for subtraction strategies. Moreover, our assay directly quantifies RNAs that correlate closely with absolute HBsAg levels (FIG. 12), without the need for intermediate steps such as library generation. The multiplex ddPCR format is therefore readily applicable and relatively inexpensive, in contrast to other platforms.

Although our tool offers insight into clinical observations, there are several limitations to this study. Our assay may not be optimized for genotypes B, G, and H, which were not represented in our cohort. We anticipate that the key findings in those genotypes would be similar, although a pangenotypic assay would be important to study global HBV. While we performed an intensive study of HBV transcription that included single-cell analyses, we only studied 16 people, all of whom were male, co-infected with HIV, and the majority were African American. In the future, we will expand our work to larger and more diverse cohorts including persons who are treatment-naïve and persons who are on stable antivirals, persons with HBV mono-infection, and females. We anticipate that while ourfindings may be consistent irrespective of which NUC is used, it will be important to apply our assay to tissues obtained from people taking novel antivirals with different mechanisms of action. We separately note that while it will be important to test how HIV affects HBV integration and transcription, HIV is unlikely to affect the overall implications of our study since the phenomenon of persistent HBsAg on NUCs is consistent between people with and without HIV infection. In addition, even if the absolute numbers of both iDNA and cccDNA-derived S are higher in people with HIV, our conclusions are based on a ratio of iDNA-:cccDNA-derived S transcripts and therefore account for higher levels of replication that can be seen in people with HIV. Another limitation is that we intentionally quantified iDNA-derived transcripts rather than iDNA specifically since our principal aim was to quantitatively link the source of S mRNA transcription and HBsAg levels.

We report a multiplex ddPCR assay that we applied to bulk liver tissue and single hepatocytes from persons with CHB, providing evidence that iDNA transcription of S is responsible for the persistent HBsAg in blood in the majority of individuals receiving long-term NUCs. Our findings have two major implications for functional HBV cure. First, therapeutically achieving a functional cure, as it is currently defined, will require durable transcriptional silencing of hepatocytes transcribing S primarily from cccDNA with NUCs or other novel agents and additionally targeting cells transcribing S primarily from iDNA, either by eliminating them or suppressing their production of S. The second implication is whether the definition of functional cure requiring complete loss of HBsAg needs to be reconsidered since it may be difficult to address all hepatocytes with iDNA.

### Methods

### Study Participants

Participants were enrolled in the HIV-HBV Cohort Ancillary Study of the Hepatitis B Research Network at Johns Hopkins Hospital. Liver biopsies were obtained from each individual at two time points separated by 2-4 years. Each biopsy was placed immediately in OCT media, flash frozen at bedside, and placed in liquid nitrogen until use. Liver tissue was sectioned in 10 microns and mounted on PEN-membrane slides for single cell laser capture microdissection (scLCM) of spatially contiguous cells in a grid to develop viral landscape maps, as previously described. For the HBeAg-negative individual, we studied twice as many cells given the relative absence of infection. Individual cells were excluded from analysis if the total RNA in a cell was not within the normal range of detection defined by greater than one standard deviation from 8 tissue-negative PEN-membrane cuts, as assayed by 7SL, to avoid analysis of cell fragments. For bulk analysis, multiple slides from each biopsy were scraped into DNA/RNA lysis buffer for extraction. Hep3B cells were obtained from the American Type Culture Collection (HB-8064).

DNA/RNA Extraction, Treatment, and ddPCR

DNA/RNA were extracted from the same sample simultaneously to limit sampling error for both bulk slide scrapes and scLCM samples, as previously described. To quantify cccDNA, half of each DNA sample was treated with Exonuclease I/Ill, as previously described, digesting all linear and partly circular DNA. Exonuclease I/III treated DNA was assayed by ddPCR using the mid-HBV assay to detect cccDNA, as previously described. DNase treated RNA samples were converted to cDNA and any remaining RNA was digested with RNase H as the last step of reverse transcription. cDNA was analyzed by ddPCR using the 5′, mid-, and 3′ HBV assays. All ddPCR runs used the following cycling parameters: one cycle of 94° C. for 10 minutes, 40 cycles of 94° C. for 30 seconds and 57° C. for one minute, one cycle of 98° C. for 10 minutes, one cycle of 12° C. for 10 minutes, and one continuous cycle at 4° C. until reading. Plates were read on the QX200 Droplet Reader (BioRad; Hercules, CA) for ddPCR.

### Transcriptional Indices

During reverse transcription, the pgRNA tail contains a direct repeat (DR) 1 that acts as a primer forming a DR1:DR2 hybrid for the viral polymerase to complete rcDNA. Approximately 10% of the time, DR1 acts as an RNA primer for another DR1 in a distinct location in the DNA to make dsIDNA. This truncation is further exacerbated when dsIDNA reenters the nucleus and is digested at its ends during non-homologous end joining at double strand DNA breaks in host chromosomes resulting in the viral genome's integration. As a result, iDNA is lacking the 5′ and 3′ ends of the HBV genome, losing promoters for all 3.5 kb transcripts including pgRNA (FIG. 10). As the promotors for 2.4, 2.1, and 0.7 kb transcripts are still present and active, these transcripts can be actively made from iDNA, though they lack the HBV polyadenylation signal (PAS) and instead rely on host PAS, forming viral-human chimeric mRNAs. By multiplexing the mid-HBV and 3′ HBV assays using ddPCR, we calculated an iDNA transcriptional index (iDNA TI) by dividing the number of transcripts originating from both iDNA and cccDNA (mid-HBV amplicon positive) to those originating solely from cccDNA (mid-HBV and 3′ HBV amplicon positive). We assumed, as has been previously reported, that the contribution of the 0.7 kb transcript (HBX) would be negligible to calculations of total HBV transcription in human tissue. Interpretations of the iDNA TI are based on comparable efficiencies of the mid-HBV and 3′ HBV assays so that a single template mRNA derived from cccDNA would be detected equivalently by these two assays. Therefore, 1) if the iDNA TI 51, transcripts are sourced from cccDNA solely, 2) if >1 and ≤2, transcripts originate from both cccDNA and iDNA, 3) if >2, the majority of transcripts originate from iDNA. There is also the special case of zero quantifiable cccDNA-derived transcription in the presence of iDNA-derived transcription and detectable cccDNA, which we interpret to mean that cccDNA transcription is fully suppressed. The pgRNA transcriptional index (pgRNA TI) was calculated, as previously published, by dividing the number of pgRNA transcripts, measured by the 5′ HBV amplicon in RNA, by the number of cccDNA molecules, quantified by the mid-HBV assay in Exonuclease I/Ill treated DNA.

### Statistics

All Spearman's correlation coefficient analyses and linear models were performed in R using “ggpubr” (28). Significance displayed as p-values was calculated using Wilcoxon rank sum and signed tests except the percent difference in transcriptionally active cells calculated by a significance test for a difference in two proportions. A p-value<0.05 was considered significant. Primer/probe fidelity analysis was performed using BLAST on Linux Ubuntu and then R, which demonstrates the ability to capture all genotypes except for F, G, and H. A tutorial for beginners on aligning oligos to HBV genotypes can be found on GitHub “HBV_primer_fidelity”.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, devices, systems, computer readable media, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1. A method of detecting and quantifying a contribution of sources of hepatitis B virus (HBV) surface antigen (HBsAg) in a sample by focusing on S transcripts (mRNAs) that are then translated into an HBsAg protein, the method comprising:
amplifying HBV transcript nucleic acids in or from the sample using at least two sets of nucleic acid primers and nucleic acid probes, wherein a first set of nucleic acid primers and nucleic acid probes targets a middle of an HBV genomic region that is also common to both HBV transcript nucleic acids transcribed from HBV covalently closed circular DNA (cccDNA) and from HBV integrated DNA (iDNA) forming mid-HBV amplicons, and wherein a second set of nucleic acid primers and nucleic acid probes targets a second portion of the HBV genomic region comprising a 3′ end of the HBV genomic region that is common to HBV transcript nucleic acids transcribed from HBV cccDNA and not from HBV iDNA forming 3′-HBV amplicons, and/or wherein a third set of nucleic acid primers and nucleic acid probes targets a third HBV genomic region comprising a 5′ end of the HBV genome that is used to distinguish full-genome transcripts from those that only target the S transcripts forming 5′-HBV amplicons to produce amplification data that comprises one or more detectable signal levels detected as the HBV transcript nucleic acids are amplified; and,
quantifying amounts of the mid-HBV amplicons and the 3′-HBV amplicons and/or the 5′-HBV amplicons from the amplification data, thereby detecting and quantifying the contribution of the sources of the HBsAg in the sample.

2. The method of claim 1, further comprising obtaining the sample from a subject.

3. The method of claim 1, wherein the sample comprises hepatocellular tissue, serum, and/or plasma.

4. The method of claim 1, comprising amplifying the HBV transcript nucleic acids in or from the sample using a multiplex droplet digital polymerase chain reaction (ddPCR) technique or multiple qPCR techniques that use the same combination of primers/probes.

5. The method of claim 1, further comprising using the method to stratify subjects into different patient populations based upon the quantified amounts of the mid-HBV amplicons and the 3′-HBV amplicons and/or the 5′-HBV amplicons for a given subject.

6. The method of claim 1, comprising administering one or more therapies to the subject based at least in part on a ratio of quantified amounts of the mid-HBV amplicons and the 3′-HBV amplicons and/or the 5′-HBV amplicons.

7. The method of claim 1, comprising discontinuing administering one or more therapies to the subject based at least in part on a ratio of the quantified amounts of the mid-HBV amplicons and the 3′-HBV amplicons and/or the 5′-HBV amplicons.

8. The method of claim 1, further comprising correlating quantified amounts of the mid-HBV amplicons and the 3′-HBV amplicons and/or the 5′-HBV amplicons with plasma levels of one or more of the HBsAg in the subject.

9. The method of claim 1, further comprising quantifying amounts of the mid-HBV amplicons and the 3′-HBV amplicons and/or the 5′-HBV amplicons from multiple samples obtained from the subject at different time points.

10. The method of claim 1, wherein the sets of nucleic acid primers and nucleic acid probes are selected from the group consisting of: a set of nucleic acids that targets a 5′ end of pre-genomic HBV RNA forming the 5′-HBV amplicons; a set of nucleic acids that targets all types of HBV transcript nucleic acids forming the mid-HBV amplicons; and a set of nucleic acids that targets a 3′ end of HBV transcript nucleic acids that are common to all HBV transcript nucleic acids that are transcribed from cccDNA forming the 3′-HBV amplicons.

11. The method of claim 1, wherein the sets of nucleic acid primers and nucleic acid probes are selected from the group consisting of: a set of nucleic acids having nucleotide sequences of SEQ ID Nos: 1, 2, and 3; a set of nucleic acids having nucleotide sequences of SEQ ID Nos: 4, 5, and 6; and a set of nucleic acids having nucleotide sequences of SEQ ID Nos: 7, 8, and 9.

12. The method of claim 1, wherein the subject has chronic HBV infection and wherein a detected decline in total quantitative HBsAg (qHBsAg) of >0.5 log 10 international units/mL in the subject indicates that the HBV transcript nucleic acids are transcribed primarily from the HBV cccDNA.

13. The method of claim 1, wherein the subject has chronic HBV infection and wherein an absence of a detected decline in total quantitative HBsAg (qHBsAg) in the subject indicates that the HBV transcript nucleic acids are transcribed primarily from the HBV iDNA.

14. A system, comprising:
a nucleic acid amplification component configured to amplify HBV transcript nucleic acids in or from a sample using at least two sets of nucleic acid primers and nucleic acid probes, wherein a first set of nucleic acid primers and nucleic acid probes targets a middle of an HBV genomic region that is also common to both HBV transcript nucleic acids transcribed from HBV covalently closed circular DNA (cccDNA) and from HBV integrated DNA (iDNA) forming mid-HBV amplicons, and wherein a second set of nucleic acid primers and nucleic acid probes targets a second portion of the HBV genomic region comprising a 3′ end of the HBV genomic region that is common to HBV transcript nucleic acids transcribed from HBV cccDNA and not from HBV iDNA forming 3′-HBV amplicons, and/or wherein a third set of nucleic acid primers and nucleic acid probes targets a third HBV genomic region comprising a 5′ end of the HBV genome that is used to distinguish full-genome transcripts from those that only target the S transcripts forming 5′-HBV amplicons to produce amplification data that comprises one or more detectable signal levels detected as the HBV transcript nucleic acids are amplified; and,
a controller operably connected to the nucleic acid amplification component, which controller comprises, or is capable of accessing, computer readable media comprising non-transitory computer-executable instructions which, when executed by at least one electronic processor, perform at least:
quantifying amounts of the mid-HBV amplicons and the 3′-HBV amplicons and/or the 5′-HBV amplicons from the amplification data to thereby detect and quantify sources of hepatitis B virus (HBV) surface antigen (HBsAg) in the sample.

15. The system of claim 14, wherein the sample comprises hepatocellular tissue, serum, and/or plasma.

16. The system of claim 14, wherein the nucleic acid amplification component is configured to perform a droplet digital polymerase chain reaction (ddPCR) technique or qPCR technique.

17. The system of claim 14, wherein the computer-executable instructions which, when executed by the at least one electronic processor, further perform at least:
stratifying subjects into different patient populations based upon the amounts of the mid-HBV amplicons and the 3′-HBV amplicons and/or the 5′-HBV amplicons quantified for a given subject.

18. The system of claim 14, wherein the computer-executable instructions which, when executed by the at least one electronic processor, further perform at least:
generating a report recommending administering one or more therapies to the subject based at least in part on a ratio of the quantified amounts of the mid-HBV amplicons and the 3′-HBV amplicons and/or the 5′-HBV amplicons.

19. The system of claim 14, wherein the computer-executable instructions which, when executed by the at least one electronic processor, further perform at least:
generating a report recommending discontinuing administering one or more therapies to the subject based at least in part on a ratio of the quantified amounts of the mid-HBV amplicons and the 3′-HBV amplicons and/or the 5′-HBV amplicons.

20. The system of claim 14, wherein the computer-executable instructions which, when executed by the at least one electronic processor, further perform at least:
correlating the quantified amounts of the mid-HBV amplicons and the 3′-HBV amplicons and/or the 5′-HBV amplicons with plasma levels of one or more of the HBsAgs in the subject.

21. The system of claim 14, wherein the sets of nucleic acid primers and nucleic acid probes are selected from the group consisting of: a set of nucleic acids that targets a 5′ end of pre-genomic HBV RNA forming the 5′-HBV amplicons; a set of nucleic acids that targets all types of HBV transcript nucleic acids forming the mid-HBV amplicons; and a set of nucleic acids that targets a 3′ end of HBV transcript nucleic acids that are common to all HBV transcript nucleic acids that are transcribed from cccDNA forming the 3′-HBV amplicons.

22. The system of claim 14, wherein the sets of nucleic acid primers and nucleic acid probes are selected from the group consisting of: a set of nucleic acids having nucleotide sequences of SEQ ID Nos: 1, 2, and 3; a set of nucleic acids having nucleotide sequences of SEQ ID Nos: 4, 5, and 6; and a set of nucleic acids having nucleotide sequences of SEQ ID Nos: 7, 8, and 9.
